(19) United States
(12) Patent Application Publication
(10) Pub. No.: US 2015/0216865 A1
(43) Pub. Date: Aug. 6, 2015

(54) METHODS OF TREATING A BRUTON'S TYROSINE KINASE DISEASE OR DISORDER

(71) Applicant: Celgene Avilomics Research, Inc., Bedford, MA (US)

(72) Inventors: William Frederick Westlin, III, Boxborough, MA (US); Erica Evans Raab, Arlington, MA (US); Zebulun David Horowitz, Basking Ridge, NJ (US); Claudia Eve Kasserra, Morris Plains, NJ (US); Lisa Beebe, Acton, MA (US); Maria Palmisano, North Chatham, NH (US); Gondi Nagendra Kumar, Basking Ridge, NJ (US); Juswinder Singh, Ashland, MA (US); Russell C. Petter, Stow, MA (US); Richland Wayne Tester, Marlborough, MA (US); Arthur F. Kluge, Lincoln, MA (US); Hormoz Mazdiyasni, Marlborough, MA (US); Deqiang Niu, Lexington, MA (US); Lixin Qiao, Tewksbury, MA (US)

(21) Appl. No.: 14/425,499

(22) PCT Filed: Sep. 3, 2013
(86) PCT No.: PCT/US2013/057880

§ 371 Date: Mar. 3, 2015

Related U.S. Application Data
(60) Provisional application No. 61/816,645, filed on Apr. 26, 2013.
(60) Provisional application No. 61/696,702, filed on Sep. 4, 2012.

(51) Int. Cl. A61K 31/505; A61K 47/10

(52) U.S. Cl. CPC A61K 31/505; A61K 47/10

(57) ABSTRACT

The present invention provides methods of treating, stabilizing or lessening the severity or progression of a disease or disorder associated with BTK.

### CROSS REFERENCE TO RELATED APPLICATIONS

The present application claims priority to U.S. provisional application Ser. No. 61/696,702, filed Sep. 4, 2012, and U.S. provisional application Ser. No. 61/816,645, filed Apr. 26, 2013, the entirety of each of which is hereby incorporated by reference.

### FIELD OF THE INVENTION

The present invention provides methods of treating, stabilizing or lessening the severity or progression of a disease or disorder associated with Bruton's Tyrosine Kinase (“BTK”).

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

The present invention provides methods of treating, stabilizing or lessening the severity or progression of one or more diseases and conditions associated with BTK. In some aspects, the present invention provides methods of treating, stabilizing or lessening the severity or progression of one or more diseases and conditions associated with BTK comprising administering to a patient in need thereof a pharmaceutically acceptable composition comprising N-(3-(5-fluoro-2-(4-(2-methoxyethoxyl)phenylamino)pyrimidin-4-ylamino)phenyl)acrylamide (1):

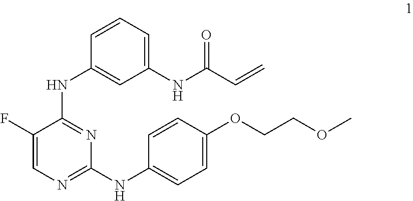 [SMILES: C=CC(=O)Nc1cccc(Nc2nc(Nc3ccc(OCCOC)cc3)ncc2F)c1]

or a pharmaceutically acceptable salt thereof.

In some embodiments, the present invention provides a method of treating, stabilizing or lessening the severity or progression of an arthritic condition, wherein the method comprises administering to a patient in need thereof a pharmaceutically acceptable composition comprising Compound 1, or a pharmaceutically acceptable salt thereof. In some embodiments, the arthritic condition is selected from osteoarthritis, rheumatoid arthritis, fibromyalgia, gout, ankylosing spondylitis, scleroderma, psoriatic arthritis, Sjogren's syndrome, Still's disease, Paget's disease, myositis, Lyme disease and juvenile idiopathic arthritis. In some embodiments, the arthritic condition is rheumatoid arthritis.

In some embodiments, provided methods comprise orally administering to a patient compositions comprising Compound 1, or a pharmaceutically acceptable salt thereof. In some embodiments, such compositions are capsule formulations. In general, provided methods comprise administering a composition which comprises Compound 1, or a pharmaceutically acceptable salt thereof, and one or more pharmaceutically acceptable excipients, such as, for example, binders, diluents, disintegrants, wetting agents, lubricants and adsorbents.

In some embodiments, the present invention also provides dosing regimens and protocols for the administration of Compound 1, or a pharmaceutically acceptable salt thereof, to patients in need thereof.

### BRIEF DESCRIPTION OF THE DRAWINGS

FIG. 1A presents a representative immunoblot of Compound 1 concentration dependent silencing of Btk activity in Ramos cells.

FIG. 1B presents a representative immunoblot of prolonged duration of action after α-IgM stimulation of the B cell receptor in Ramos cells (n=3 experiments).

FIG. 2 is a schematic depicting the direct assessment of Btk occupancy utilizing a covalent probe. Covalent probe Compound 2 detects free, uninhibited Btk in lysates derived from tissue culture, animal tissues or clinical samples. Samples treated with Compound 1 are lysed and then incubated with 1 μM Compound 2. Uninhibited Btk in the lysate is captured by Compound 2 and quantitated by streptavidin (SA)-coated ELISA plate. Normalization to untreated control sample allows determination of the % Btk occupancy.

FIG. 3A presents an immunoblot of Compound 1 concentration dependent silencing of Btk activity in human primary B cells.

FIG. 3B presents a graph depicting concentration dependent % Btk occupancy of Compound 1 in human primary B cells.

FIG. 4 presents kinase inhibition of Compound 1 in a human primary B cell proliferation assay.

FIG. 5 presents a graph depicting the Btk occupancy and plasma levels of Compound 1 for >12 hours after circulating compound has disappeared.

FIG. 6A presents the dose-dependent inhibition of disease symptoms as measured by the daily clinical arthritis score plotted over 14 days of treatment in an established collagen-induced arthritis Dba1 mouse model. At either 10 or 30 mg/kg, Compound 1 besylate was similar to dexamethasone control in inhibiting disease symptoms. **p<0.05 for 10 and 30 mg/kg Compound 1 (ANOVA).

FIG. 6B presents the Btk occupancy of Compound 1 besylate. At 3 mg/kg, 34% Btk occupancy was observed at 2 hours; at 10 and 30 mg/kg, 84-95% Btk occupancy was observed at 2 hours, respectively. Occupancy shown for each individual mouse with mean of each group indicated by bar.

FIG. 6C presents a histopathologic analysis of the six joints in affected CIA mice demonstrated decreased cartilage and bone damage as well as inflammation and pannus in Compound 1 besylate-treated animals. Compound 1's inhibition of histopathologic signs of disease was significant (p<0.05) and dose-dependent such that 10 and 30 mg/kg had effects similar to that of the positive control dexamethasone (Inhibition 82% with dexamethasone, 87% with 10 mg/kg Compound 1, 96% with 30 mg/kg Compound 1 besylate).

FIG. 7 presents the clinical arthritis score for semi-established CIA mice.

FIG. 8 presents PK and PD analysis of 6 human subjects dosed with Compound 1 besylate. Compound 1 besylate was rapidly absorbed, with mean peak plasma levels (Cmax) of 542 ng/mL. PD analysis of Btk target occupancy in the same 6 subjects displayed maximal occupancy at 4 hours (average cohort occupancy >97%) with sustained occupancy through 24 hours and recovery of Btk protein levels towards 50% 48-72 hours after Compound 1 besylate administration. Mean±SEM for plasma level and % Btk occupancy depicted.

FIG. 9 presents the mean rear ankle thickness in PGPS rats.

FIG. 10 presents a graph depicting the Btk target site occupancy ELISA used to detect free Btk protein from Ramos cells treated with increasing concentrations of Compound 1 (0.3 nM-3 μM) for one hour to determine the remaining pg free Btk/μg total protein at each concentration. Values were normalized to DMSO-treated control samples to obtain % occupancy. The EC₅₀ of Compound 1 as measured by occupancy ELISA was 6 nM, EC₉₀ equaling 39 nM (mean±SD of n=4 experiments shown).

FIG. 11 presents a graph depicting the body weight loss associated with collagen-induced arthritis.

FIG. 12 presents a graph depicting the body weight in grams (mean±SEM) of mice in a non-obese diabetic mouse model of Sjodren's syndrome.

FIG. 13 presents a graph depicting the body weight as a percentage of initial bodyweight (mean±SEM) of mice in a non-obese diabetic mouse model of Sjodren's syndrome.

FIG. 14 presents a graph depicting the lachrymal gland secretion (mean±SEM) of mice in a non-obese diabetic mouse model of Sjodren's syndrome.

FIG. 15 presents a bar graph depicting the lachrymal gland secretion (mean±SEM) of mice at 20 weeks of age in a non-obese diabetic mouse model of Sjodren's syndrome.

FIG. 16 presents a graph depicting the salivary gland secretion (mean±SEM) of mice in a non-obese diabetic mouse model of Sjodren's syndrome.

FIG. 17 presents a bar graph depicting the histopathology Focus Score (mean±SEM) of lachrymal glands from control BALB/c and vehicle-treated NOD mice.

FIG. 18 presents a bar graph depicting the histopathology Area Score (mean±SEM) of lachrymal glands from control BALB/c and vehicle-treated NOD mice.

FIG. 19 presents a bar graph depicting the histopathology Focus Score (mean±SEM) of salivary glands from control BALB/c and vehicle-treated NOD mice.

FIG. 20 presents a bar graph depicting the histopathology Area Score (mean±SEM) of salivary glands from control BALB/c and vehicle-treated NOD mice.

### DETAILED DESCRIPTION OF THE INVENTION

### Definitions

As used herein, a “disease or disorder associated with BTK” means any disease or other deleterious condition in which BTK, or a mutant thereof, is known or suspected to play a role. Accordingly, another embodiment of the present invention relates to preventing, treating, stabilizing or lessening the severity or progression of one or more diseases in which BTK, or a mutant thereof, is known or suspected to play a role. Specifically, the present invention relates to a method of treating or lessening the severity of a proliferative disorder, wherein said method comprises administering to a patient in need thereof Compound 1, or a pharmaceutically acceptable salt thereof, or a pharmaceutically acceptable composition thereof.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with Bruton's tyrosine kinase.

The term “subject”, as used herein, means a mammal and includes human and animal subjects, such as domestic animals (e.g., horses, dogs, cats, etc.).

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of inventive formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

### Compound 1 is an Irreversible BTK Inhibitor

United States published patent application number US 2010/0029610, published Feb. 4, 2010 (“the '610 publication,” the entirety of which is hereby incorporated herein by reference), describes certain 2,4-disubstituted pyrimidine compounds which covalently and irreversibly inhibit activity of one or more protein kinases, including BTK, a member of TEC-kinases. Such compounds include Compound 1, which is designated as compound number I-182 in the '610 publication. The synthesis of Compound 1 is described in detail at Example 20. Compound 1 is active in a variety of assays and therapeutic models demonstrating covalent, irreversible inhibition of BTK (in enzymatic and cellular assays). Accordingly, Compound 1 is useful for treating one or more disorders associated with activity of BTK.

The present invention provides methods of treating, stabilizing or lessening the severity or progression of one or more diseases and conditions associated with BTK comprising administering to a patient in need thereof a pharmaceutically acceptable composition comprising Compound 1, or a pharmaceutically acceptable salt thereof, wherein the pharmaceutically acceptable composition is an oral dosage form. In some such embodiments, the pharmaceutically acceptable composition is formulated as a capsule. Such methods, dosing regimens and protocols for the administration of pharmaceutically acceptable compositions comprising Compound 1, or a pharmaceutically acceptable salt thereof, are described in further detail, below.

### I. General Dosing Protocol

As described above, the present invention provides methods of treating, stabilizing or lessening the severity or progression of one or more diseases or conditions associated with BTK, wherein the method comprises administering to a patient in need thereof a pharmaceutically acceptable composition comprising Compound 1, or a pharmaceutically acceptable salt thereof. In some embodiments, the present invention provides a method of preventing the progression of a disease or disorder associated with BTK. It is understood that although the methods described herein refer to administering Compound 1, such methods are equally applicable to methods of administering a salt form of Compound 1, e.g., a besylate salt of Compound 1. Accordingly, methods provided herein are to be understood to encompass either the administration of Compound 1 or a pharmaceutically acceptable salt thereof.

In some embodiments, provided methods comprise administering to a patient in need thereof a pharmaceutically acceptable composition comprising from about 5% to about 60% of Compound 1, based upon total weight of the formulation. In some embodiments, provided methods comprise administering to a patient in need thereof a pharmaceutically acceptable composition comprising from about 5% to about 15% or about 7% to about 15% or about 7% to about 10% or about 9% to about 12% of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of Compound 1, based upon total weight of the formulation. In certain embodiments, provided methods comprise administering to a patient in need thereof a pharmaceutically acceptable composition comprising from about 6%, about 7%, about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of Compound 1, based upon total weight of given composition or formulation.

In some embodiments, provided methods comprise administering to a patient in need thereof a pharmaceutically acceptable composition comprising from about 25% to about 75% or about 30% to about 60% or about 40% to about 50% or about 40% to about 45% of Compound 1, or a pharmaceutically acceptable salt thereof, based upon total weight of the formulation. In some embodiments, provided methods comprise administering to a patient in need thereof a pharmaceutically acceptable composition comprising from about 6%, about 7%, about 8%, about 9%, about 10%, about 11%, about 12%, about 13% of Compound 1, or a pharmaceutically acceptable salt thereof, based upon total weight of given composition or formulation. In certain embodiments, provided methods comprise administering to a patient in need thereof a pharmaceutically acceptable composition comprising from about 41%, about 42%, about 43%, about 44% or about 45%, of Compound 1, or a pharmaceutically acceptable salt thereof, based upon total weight of given composition or formulation.

In some embodiments, provided methods comprise administering a pharmaceutically acceptable composition comprising Compound 1 one, two, three, or four times a day. In some embodiments, a pharmaceutically acceptable composition comprising Compound 1 is administered once daily (“QD”). In some embodiments, a pharmaceutically acceptable composition comprising Compound 1 is administered twice daily. In some embodiments, twice daily administration refers to a compound or composition that is administered “BID”. A “BID” dose is a particular dose (e.g., a 125 mg dose) that is administered twice a day (i.e., two doses of 125 mg administered at two different times in one day). In some embodiments, twice daily administration refers to a compound or composition that is administered in two different doses, wherein the first administered dose differs from the second administered dose. For example, a 250 mg dose administered twice daily can be administered as two separate doses, one 150 mg dose and one 100 mg dose, wherein each dose is administered at a different time in one day. Alternatively, a 250 mg dose administered twice daily can be administered 125 mg BID (i.e., two 125 mg doses administered at different times in one day). In some embodiments, a total daily dose of 375 mg of Compound 1 can be administered as a 250 mg dose administered at a given timepoint (for example, in the morning) and a 125 mg dose administered at a later timepoint (for example, in the evening).

In some embodiments, a pharmaceutically acceptable composition comprising Compound 1 is administered three times a day. In some embodiments, a pharmaceutically acceptable composition comprising Compound 1 is administered “TID”, or three equivalent doses administered at three different times in one day. In some embodiments, a pharmaceutically acceptable composition comprising Compound 1 is administered in three different doses, wherein at least one of the administered doses differs from another administered dose. In some embodiments, a pharmaceutically acceptable composition comprising Compound 1 is administered four times a day. In some embodiments, a pharmaceutically acceptable composition comprising Compound 1 is administered “QID”, or four equivalent doses administered at four different times in one day. In some embodiments, a pharmaceutically acceptable composition comprising Compound 1 is administered in four different doses, wherein at least one of the administered doses differs from another administered dose.

In some embodiments, Compound 1 is administered to a patient twice a day, wherein the first administered dose differs from the second administered dose. In some such embodiments, a total daily dose of 375 mg of Compound 1 can be administered as a 250 mg dose administered at a given timepoint (for example, in the morning) and a 125 mg dose administered at a later timepoint (for example, in the evening).

In some embodiments, provided methods comprise administering a pharmaceutically acceptable composition comprising Compound 1 once a day (“QD”). In some embodiments, provided methods comprise administering a pharmaceutically acceptable composition comprising Compound 1 twice a day. In some embodiments, a pharmaceutically acceptable composition comprising Compound 1 is administered once or twice daily for a period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a pharmaceutically acceptable composition comprising Compound 1 is administered once or twice daily for 28 consecutive days (“a 28-day cycle”). In some embodiments, a pharmaceutically acceptable composition comprising Compound 1 is administered once or twice daily for at least one 28-day cycle. In some embodiments, a pharmaceutically acceptable composition comprising Compound 1 is administered once or twice daily for at least two, at least three, at least four, at least five or at least six 28-day cycles. In some embodiments, a pharmaceutically acceptable composition comprising Compound 1 is administered once or twice daily for at least seven, at least eight, at least nine, at least ten, at least eleven or at least twelve 28-day cycles. In some embodiments, a pharmaceutically acceptable composition comprising Compound 1 is administered once or twice daily for at least thirteen, at least fourteen, at least fifteen, at least sixteen, at least seventeen, at least eighteen, at least nineteen or at least twenty 28-day cycles. In some embodiments, a pharmaceutically acceptable composition comprising Compound 1 is administered to a patient for the duration of the patient's life.

In some embodiments, two adjacent 28-day cycles may be separated by a rest period. Such a rest period may be one, two, three, four, five, six, seven or more days during which the patient is not administered a unit dose of Compound 1. In a preferred embodiment, two adjacent 28-day cycles are continuous.

### Unit Dosage Forms

Pharmaceutical compositions for use in the present invention may be prepared as a unit dosage form. A person of ordinary skill will appreciate that the unit dosage forms described herein refer to an amount of Compound 1 as a free base. A person skilled in the art will further appreciate that, when a pharmaceutical composition comprises a salt form of Compound 1, for example a besylate salt form, the amount of the salt form present in the composition is an amount that is equivalent to a unit dose of the free base of Compound 1. For example, a pharmaceutical composition comprising a besylate salt of Compound 1 would contain 34.97 mg of the besylate salt form necessary to deliver an equivalent 25 mg unit dose of the free base of Compound 1.

In some embodiments, provided methods comprise administering to a patient in need thereof a composition comprising a unit dose of Compound 1, wherein the unit dose is about 75 mg to about 750 mg. In some embodiments, provided methods comprise administering to a patient in need thereof a composition comprising a unit dose of Compound 1, wherein the unit dose is about 125 mg to about 750 mg. In some embodiments, provided methods comprise administering to a patient in need thereof a composition comprising a unit dose of Compound 1, wherein the unit dose is about 125 mg to about 500 mg. In some embodiments, provided methods comprise administering to a patient in need thereof a composition comprising a unit dose of Compound 1, wherein the unit dose is about 250 mg to about 500 mg. In some embodiments, a unit dose of Compound 1 is administered once a day (QD). In some embodiments, a unit dose of Compound 1 is administered twice a day. In some embodiments, a unit dose of Compound 1 is administered BID.

In some embodiments, the unit dose of Compound 1 is about 25 mg to 750 mg, or about 25 mg to about 625 mg, or about 25 mg to about 500 mg, or about 25 mg to about 375 mg, or about 25 mg to about 250 mg, or about 25 mg to about 125 mg, or about 25 mg to about 75 mg, or about 75 mg to about 750 mg, or about 75 mg to about 625 mg, or about 75 mg to about 500 mg, or about 75 mg to about 375 mg, or about 75 mg to about 250 mg, or about 75 mg to about 125 mg, or about 125 mg to about 750 mg, or about 125 mg to about 625 mg, or about 125 mg to about 500 mg, or about 125 mg to about 375 mg, or about 125 mg to about 250 mg, or about 250 mg to about 750 mg, or about 250 mg to about 625 mg, or about 250 mg to about 500 mg, or about 250 mg to about 375 mg, or about 375 mg to about 750 mg, or about 375 mg to about 625 mg, or about 375 mg to about 500 mg, or about 500 mg to about 750 mg, or about 500 mg to about 625 mg, or about 625 mg to about 750 mg.

In some embodiments, the unit dose of Compound 1 is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, Compound 1 is administered two, three or four times a day, wherein each dose is identical. In some embodiments, Compound 1 is administered two, three or four times a day, wherein at least one dose is different from another dose. In some such embodiments, each dose may be independently selected from those doses or dose ranges in the two preceeding paragraphs.

In some embodiments, provided methods comprise administering to a patient in need thereof a pharmaceutical composition comprising a unit dose of Compound 1. In some such embodiments, the unit dose is about 25 mg, about 50 mg, about 75 mg, about 100 mg, about 125 mg, about 150 mg, about 175 mg, about 200 mg, about 225 mg or about 250 mg.

### II. Uses of Compounds and Pharmaceutically Acceptable Compositions

Compound 1 and compositions described herein are generally useful for the inhibition of protein kinase activity of one or more enzymes. Examples of kinases that are inhibited by Compound 1 and compositions described herein and against which the methods described herein are useful include BTK and other TEC-kinases, including ITK, TEC, BMX and RLK, or a mutant thereof.

Bruton's tyrosine kinase (Btk) is a member of the Tec family of cytosolic tyrosine kinases and is expressed exclusively in B cells and cells of the myeloid lineage. Btk has a well characterized essential role in B cells that is highlighted by the human primary immune deficiency disease, X-linked agammaglobulinemia (XLA), which results from mutation in the Btk gene and produces a functionally inactive protein (Smith et al., “X-linked agammaglobulinemia: lack of mature B lineage cells caused by mutations in the Btk kinase,” Springer Semin. Immunopathol. 1998, 19:369-381). XLA patients display a B cell differentiation block at the pro-B to pre-B cell transition (Campana et al., “Phenotypic features and proliferative activity of B cell progenitors in X-linked agammaglobulinemia,” J. Immunol. 1990, 145:1675-1680). As a result of incomplete B cell differentiation, these patients have a near complete absence of mature B cells in the peripheral blood (Campana et al., “Phenotypic features and proliferative activity of B cell progenitors in X-linked agammaglobulinemia,” J. Immunol. 1990, 145:1675-1680) and cannot produce immunoglobulins of any class (Conley, “B cells in patients with X-linked agammaglobulinemia,” J. Immunol. 1985, 134:3070-3074; Nonoyama et al., “Functional analysis of peripheral blood B cells in patients with X-linked agammaglobulinemia,” J. Immunol. 1998, 161:3925-3929). Furthermore, the human XLA phenotype is recapitulated, although less severely, in Btk knock-out mice (Khan et al., “Defective B cell development and function in Btk-deficient mice,” Immunity. 1995, 3:283-299) and in xid mice which have a naturally occurring Btk mutation (Rawlings et al., “Mutation of unique region of Bruton's tyrosine kinase in immunodeficient XID mice,” Science 1993, 261:358-361). These Btk deficient mice display a 50% reduction in circulating B-2 cells, an absence of CD5+ B-1 cells and a failure to respond to T cell independent type II antigens (Rawlings et al., “Mutation of unique region of Bruton's tyrosine kinase in immunodeficient XID mice,” Science 1993, 261:358-361; Wicker et al., “X-linked immune deficiency (xid) of CBA/N mice,” Curr. Top. Microbiol. Immunol. 1986, 124:87-101; Sideras et al., “Molecular and cellular aspects of X-linked agammaglobulinemia,” Adv. Immunol. 1995, 59:135-223; Satterthwaite et al., “Btk dosage determines sensitivity to B cell antigen receptor cross-linking,” Proc. Natl. Acad. Sci. U.S.A 1997, 94:13152-13157) demonstrating a requirement for Btk in normal B cell development and function.

Specifically, Btk plays an essential role in the B cell receptor (BCR) signaling pathway. Antigen binding of the BCR results in B cell receptor oligomerization, leading to interaction of Syk and Lyn kinases with aggregated immunoreceptor tyrosine-based activation motifs (ITAMS) on the CD79 subunit of the BCR and subsequent phosphorylation and activation (Gauld et al., “B cell antigen receptor signaling: roles in cell development and disease,” Science 2002, 296:1641-1642). Lyn and Syk phosphorylate Btk on Tyr551 resulting in activation of the kinase (Rawlings et al., “Activation of BTK by a phosphorylation mechanism initiated by SRC family kinases,” Science 1996, 271:822-825; Park et al., “Regulation of Btk function by a major autophosphorylation site within the SH3 domain,” Immunity 1996, 4:515-525; Baba et al., “BLNK mediates Syk-dependent Btk activation,” Proc. Natl. Acad. Sci. U.S.A 2001, 98:2582-2586). Once activated, Btk translocates to the lipid membrane where it forms a signaling complex with proteins such as Blnk, Lyn, and Syk and phosphorylates PLCγ2 (Baba et al., “BLNK mediates Syk-dependent Btk activation,” Proc. Natl. Acad. Sci. U.S.A 2001, 98:2582-2586; Tsukada et al., “Btk and BLNK in B cell development. Adv. Immunol. 2001, 77:123-162). This in turn leads to downstream release of intracellular Ca²⁺ stores and propagation of the BCR signaling pathway through Erk and Nf-κB signaling that ultimately results in transcriptional changes to foster B cell survival, proliferation, and/or differentiation (Baba et al., “BLNK mediates Syk-dependent Btk activation,” Proc. Natl. Acad. Sci. U.S.A 2001, 98:2582-2586; Maas et al., “Role of Bruton's tyrosine kinase in B cell development,” Dev. Immunol. 2001, 8:171-181; Mohamed et al., “Bruton's tyrosine kinase (Btk): function, regulation, and transformation with special emphasis on the PH domain,” Immunol. Rev. 2009, 228:58-73).

While essential in the normal development and function of B cells, there are several pathologies that have been attributed in part to dysregulated BCR activity. The expression of Btk is highly restricted to cells of hematopoietic lineage including B lymphocytes, mast cells, monocytes, and osteoclasts. This highly restricted expression pattern of Btk together with the prominent role of Btk in the BCR signaling pathway makes it an attractive drug target for the treatment of B cell-associated autoimmune diseases. These include diseases of autoreactivity such as that observed in lupus, multiple sclerosis (MS), and rheumatoid arthritis (RA) in which B cells inappropriately break self-tolerance to produce autoantibodies and contribute to autoimmune disease (Edwards et al., “B-cell targeting in rheumatoid arthritis and other autoimmune diseases,” Nat. Rev. Immunol. 2006, 6:394-403; Teng et al., “Targeted therapies in rheumatoid arthritis: Focus on rituximab,” Biologics 2007, 1:325-333; Edwards et al., “Prospects for B-cell-targeted therapy in autoimmune disease,” Rheumatology (Oxford) 2005, 44:151-156). It has also been recently recognized that BCR signaling contributes to several B cell malignancies such as chronic lymphocytic leukemia (CLL) (Chen et al., “ZAP-70 directly enhances IgM signaling in chronic lymphocytic leukemia,” Blood 2005, 105:2036-2041; Hoellenriegel et al., “The phosphoinositide 3′-kinase delta inhibitor, CAL-101, inhibits B-cell receptor signaling and chemokine networks in chronic lymphocytic leukemia,” Blood 2011, 118:3603-3612; Stevenson et al., “B-cell receptor signaling in chronic lymphocytic leukemia,” Blood 2011, 118:4313-4320), mantle cell leukemia (MCL) and subsets of diffuse large B cell lymphoma (DLBCL) (Suljagic et al., “The Syk inhibitor fostamatinib disodium (R788) inhibits tumor growth in the Emu-TCL1 transgenic mouse model of CLL by blocking antigen-dependent B-cell receptor signaling,” Blood 2010, 116:4894-4905; Chen et al., “SYK-dependent tonic B-cell receptor signaling is a rational treatment target in diffuse large B-cell lymphoma,” Blood 2008, 111:2230-2237; Davis et al., “Chronic active B-cell-receptor signalling in diffuse large B-cell lymphoma,” Nature 2010, 463:88-92; Lenz et al., “Molecular subtypes of diffuse large B-cell lymphoma arise by distinct genetic pathways,” Proc. Natl. Acad. Sci. U.S.A 2008, 105:13520-13525; Pighi et al., “Phospho-proteomic analysis of mantle cell lymphoma cells suggests a pro-survival role of B-cell receptor signaling,” Cell Oncol. (Dordr.) 2011, 34:141-153; Baran-Marszak et al., “Constitutive and B-cell receptor-induced activation of STAT3 are important signaling pathways targeted by bortezomib in leukemic mantle cell lymphoma,” Haematologica 2010, 95:1865-1872). However, until recently, therapies that target the B cell have resulted in depletion of the B cell repertoire while therapeutic strategies that reduce BCR activity are relatively new for treatment of these diseases. Promising recent clinical data generated by inhibition of distinct BCR signaling components, including Syk and PI3Kδ with fostamatinib and GS-1101(CAL-101), respectively, have emerged providing great excitement for this approach. Inhibition of Syk with fostamatinib has demonstrated efficacy in preclinical models of inflammatory disease and in human clinical trials in autoimmune diseases (RA and ITP) as well as in B cell malignancies dependent on BCR signaling such as CLL (Chen et al., “SYK-dependent tonic B-cell receptor signaling is a rational treatment target in diffuse large B-cell lymphoma,” Blood 2008, 111:2230-2237; Friedberg et al., “Inhibition of Syk with fostamatinib disodium has significant clinical activity in non-Hodgkin lymphoma and chronic lymphocytic leukemia,” Blood 2010, 115:2578-2585; Genovese et al., “An oral Syk kinase inhibitor in the treatment of rheumatoid arthritis: A three-month randomized, placebo-controlled, phase II study in patients with active rheumatoid arthritis that did not respond to biologic agent,” Arthritis Rheum. 2011, 63:337-345; Podolanczuk et al., “Of mice and men: an open-label pilot study for treatment of immune thrombocytopenic purpura by an inhibitor of Syk,” Blood 2009, 113:3154-3160; Braselmann et al., “R406, an orally available spleen tyrosine kinase inhibitor blocks fc receptor signaling and reduces immune complex-mediated inflammation,” J. Pharmacol. Exp. Ther. 2006, 319:998-1008).

Similarly, inhibition of PI3Kδ with GS-1101 has also shown promising results in CLL (Hoellenriegel et al., “The phosphoinositide 3′-kinase delta inhibitor, CAL-101, inhibits B-cell receptor signaling and chemokine networks in chronic lymphocytic leukemia,” Blood 2011, 118:3603-3612; Lannutti et al., “CAL-101, a p110delta selective phosphatidylinositol-3-kinase inhibitor for the treatment of B-cell malignancies, inhibits PI3K signaling and cellular viability,” Blood 2011, 117:591-594; Herman et al., “Phosphatidylinositol 3-kinase-delta inhibitor CAL-101 shows promising preclinical activity in chronic lymphocytic leukemia by antagonizing intrinsic and extrinsic cellular survival signals,” Blood 2010, 116:2078-2088). Btk, downstream of Syk and PI3Kδ in the BCR signaling pathway, also represents an attractive drug target in diseases characterized by aberrant B cell activity. Moreover, owing to its highly restricted expression pattern in B cells and myeloid cells, Btk provides an opportunity for selective therapeutic targeting. Preclinically, small molecule inhibition of Btk with CGI1746 and PCI-32765 demonstrated therapeutic activity in several models of autoimmune disease (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080; Chang et al., “The Bruton tyrosine kinase inhibitor PCI-32765 ameliorates autoimmune arthritis by inhibition of multiple effector cells,” Arthritis Res. Ther. 2011, 13:R115; Di Paolo et al., “Specific Btk inhibition suppresses B cell- and myeloid cell-mediated arthritis,” Nat. Chem. Biol. 2011, 7:41-50). PCI-32765 has demonstrated initial anti-tumor activity against B cell lymphomas in canines (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080) and is showing promising results in early clinical development for the treatment of B cell malignancies (Harrison, “Trial watch: BTK inhibitor shows positive results in B cell malignancies,” Nat. Rev. Drug Discov. 2012, 11:96), providing evidence that Btk represents a viable and efficacious therapeutic target.

Arthritis refers generally to more than 100 rheumatic diseases and conditions that affect joints, tissues surrounding joints and other connective tissues. Rheumatic diseases are characterized by pain and stiffness in or around one or more joints. The most common arthritic condition is osteoarthritis. Other frequently occurring arthritic conditions include rheumatoid arthritis, fibromyalgia, gout, ankylosing spondylitis, scleroderma, psoriatic arthritis, Sjogren's syndrome, Still's disease, Paget's disease, myositis, Lyme disease and juvenile idiopathic arthritis. Common symptoms of an arthritic condition include pain, aching, stiffness and swelling in or around the joints. Arthritic symptoms can develop gradually or suddenly. Certain of the rheumatic conditions can also involve the immune system (e.g., rheumatoid arthritis) and various internal organs of the body.

### Rheumatoid Arthritis

Rheumatoid arthritis (RA) is a chronic systemic autoimmune inflammatory disease characterized by persistent synovial inflammation. Such chronic inflammation can cause permanent joint destruction and deformity. While inflammation of the tissue around the joints is a characteristic feature of RA, the disease can also cause inflammation and injury in other organs of the body.

Worldwide, the annual incidence of RA is approximately 3 cases per 10,000 individuals, and the prevalence rate is approximately 1%. RA affects women three times more frequently than men with a typical onset of RA occurring between the ages of 20 to 40 years. RA affects all populations, although it is much more prevalent in some groups (eg, 5-6% in some Native American groups) and much less prevalent in others (eg, black persons from the Caribbean region).

The pathogenesis of RA is not completely understood. An external trigger (eg, infection, trauma) that causes an autoimmune reaction, leading to synovial hypertrophy and chronic joint inflammation along with the potential for extra-articular manifestations, is theorized to occur in genetically susceptible individuals. Synovial cell hyperplasia and endothelial cell activation are early events in the pathologic process that progresses to uncontrolled inflammation and consequent cartilage and bone destruction. Genetic factors and immune system abnormalities contribute to disease propagation.

CD4 T cells, mononuclear phagocytes, fibroblasts, osteoclasts, and neutrophils play major cellular roles in the pathophysiology of RA, whereas B lymphocytes produce autoantibodies (ie, rheumatoid factors [RFs]). Abnormal production of numerous cytokines, chemokines, and other inflammatory mediators (eg, tumor necrosis factor alpha [TNF-alpha], interleukin [IL]-1, IL-6, transforming growth factor beta [TGF-beta], IL-8, fibroblast growth factor [FGF], platelet-derived growth factor [PDGF]) has been demonstrated in patients with RA. Ultimately, inflammation and proliferation of synovium or “pannus” leads to destruction of various tissues, including cartilage, bone, tendons, ligaments, and blood vessels. Although the articular structures are the primary sites involved by RA, other tissues are also affected. The consequences of uncontrolled RA include joint destruction with resultant disability, increased risk of co-morbidities such as coronary artery disease, and shortened life expectancy. The effects of concurrent therapy, which is often immunosuppressive, may contribute to mortality in RA. However, studies suggest that control of inflammation may improve mortality.

The goals of RA management are to improve signs and symptoms, prevent loss of function and minimize structural joint damage, which can occur within the first 3-6 months of disease onset. While non-steroidal anti-inflammatory drugs (NSAIDS) or low-dose prednisone may improve symptoms and function by reducing inflammation, current treatment paradigms recognize the importance of early intervention with disease modifying anti-rheumatic drugs (DMARDs), since long-term preservation of functional status becomes increasingly dependent upon prevention of structural damage along with control of inflammation. Among the commonly used small molecule DMARDs, methotrexate (MTX) has become the “gold standard” based upon its overall favorable benefit to risk ratio, long-term tolerability, favorable effects on radiographic progression of disease and positive impact on mortality. However, traditional DMARDs such as MTX monotherapy rarely induce remission of the disease and more than half of these patients do not obtain a clinically meaningful response.

Recent advances in molecular technology have guided the development of biologic DMARDs targeting cytokines, cytokine receptors, B lymphocytes and co-stimulatory pathways. The biologic DMARDs, alone or in combination with traditional DMARDs, have demonstrated significant efficacy and structural preservation superior to that observed with MTX alone. However, these agents must be administered parenterally and are associated with long-term safety concerns. In addition, up to 30% of patients have been known to fail to respond to these newer, advanced therapies. There is an unmet medical need for more effective treatments and more treatment options for RA patients based on an understanding of the underlying pathophysiology of the disease process.

### Role of B Cells in Rheumatoid Arthritis

The precise contribution of B cells to the immunopathogenesis of RA is not well characterized, although it has been suggested that several mechanisms in which B cell driven mechanisms are implicated. B cells may function as antigen presenting cells and provide important co-stimulatory signals required for CD4+ T cell clonal expansion and effector functions. In patients with RA, the synovial membrane is characterized by a prominent infiltrate of chronic inflammatory cells. These inflammatory cells, primarily CD4+ T lymphocytes, then stimulate monocytes, synovial fibroblasts, and macrophages to produce key pro-inflammatory cytokines Activated inflammatory cells proceed to release matrix metalloproteinases, eicosanoids, and toxic oxygen and nitrogen species. Activated CD4+ T cells also contribute to joint damage by stimulating the development of osteoclasts to erode bone and by stimulating B cells to produce immunoglobulins, such as rheumatoid factor. B cells have been shown to be instrumental in joint inflammation and destruction as evidenced by efficacious treatment with rituximab, an anti-CD20 B cell depleting agent. B cells play a central role in the pathophysiology of RA and therefore merit investigation as a therapeutic target. The highly restricted expression pattern of Bruton's tyrosine kinase (Btk), together with the prominent role of Btk in the B cell receptor signaling pathway makes Btk an attractive drug target for treatment of complex autoimmune disorders like RA with Compound 1.

Compound 1 is a potent, selective, orally administered small molecule inhibitor of Btk, which is an integral component of the B cell receptor signaling complex with distribution limited primarily to B lymphocytes and myeloid cells. Btk plays a crucial role in B cell development and function. Compound 1 inhibits Btk activity by binding with high affinity to the adenosine triphosphate (ATP) binding site of Btk. Compound 1 forms a covalent bond with the target Btk protein, providing rapid, complete, and prolonged/irreversible inhibition of Btk activity, both in vitro and in vivo.

Compound 1 has efficacy in an inflammatory disease model of arthritis, for example, rheumatoid arthritis. The reduced severity of disease after Btk inhibition with Compound 1 in nonclinical disease models recapitulates the phenotype seen in xid mice which harbor an inactivating mutation in the Btk gene and have been shown to have a reduced incidence and severity of collagen-induced arthritis. These observations further support Btk as a molecular target of therapeutic potential in autoimmune diseases. Thus, in some embodiments, the present invention provides a method of treating, stabilizing or lessening the severity or progression of a BTK-mediated disorder comprising the step of administering to a patient in need thereof N-(3-(5-fluoro-2-(4-(2-methoxyethoxyl)phenylamino)pyrimidin-4-ylamino)phenyl)acrylamide, or a pharmaceutically acceptable salt thereof. In some embodiments, provided methods comprise administering to a patient in need thereof a pharmaceutically acceptable composition comprising N-(3-(5-fluoro-2-(4-(2-methoxyethoxyl)phenylamino)pyrimidin-4-ylamino)phenyl)acrylamide, or a pharmaceutically acceptable salt thereof.

As used herein, the term “BTK-mediated” disorders or conditions as used herein means any disease or other deleterious condition in which BTK, or a mutant thereof, is known or suspected to play a role. Accordingly, another embodiment of the present invention relates to treating, stabilizing or lessening the severity or progression of one or more diseases in which BTK, or a mutant thereof, is known or suspected to play a role. Specifically, the present invention relates to a method of treating, stabilizing or lessening the severity or progression of an arthritic condition, wherein said method comprises administering to a patient in need thereof Compound 1, or a pharmaceutically acceptable salt thereof, or a composition according to the present invention. In some embodiments, the arthritic condition is selected from osteoarthritis, rheumatoid arthritis, fibromyalgia, gout, ankylosing spondylitis, scleroderma, psoriatic arthritis, Sjogren's syndrome, Still's disease, Paget's disease, myositis, Lyme disease and juvenile idiopathic arthritis. In some embodiments, the arthritic condition is rheumatoid arthritis.

III. Methods of Treating Diseases or Disorders Associated with BTK

In some embodiments, the present invention provides a method of treating, stabilizing or lessening the severity or progression of an arthritic condition, the method comprising administering to a patient in need thereof a therapeutically effective amount of a pharmaceutically acceptable composition comprising Compound 1, wherein the pharmaceutically acceptable composition is administered as an oral dosage form. In some such embodiments, the oral dosage form is a capsule.

In some embodiments, the present invention provides a method of treating, stabilizing or lessening the severity or progression of an arthritic condition, for example rheumatoid arthritis, the method comprising administering to a patient in need thereof a solid oral dosage form comprising a unit dose of Compound 1, wherein the unit dose is about 25 mg, about 50 mg, about 75 mg, about 100 mg, about 125 mg, about 150 mg, about 175 mg, about 200 mg, about 225 mg or about 250 mg.

In some embodiments, the present invention provides a method of treating, stabilizing or lessening the severity or progression of an arthritic condition, for example, rheumatoid arthritis, the method comprising administering to a patient in need thereof a pharmaceutical composition comprising Compound 1.

In some embodiments, the present invention provides a method of treating, stabilizing or lessening the severity or progression of an arthritic condition, for example, rheumatoid arthritis, the method comprising administering to a patient in need thereof a pharmaceutical composition comprising about 25 mg to about 750 mg, or about 25 mg to about 625 mg, or about 25 mg to about 500 mg, or about 25 mg to about 375 mg, or about 25 mg to about 250 mg, or about 25 mg to about 125 mg, or about 25 mg to about 75 mg, or about 75 mg to about 750 mg, or about 75 mg to about 625 mg, or about 75 mg to about 500 mg, or about 75 mg to about 375 mg, or about 75 mg to about 250 mg, or about 75 mg to about 125 mg, or about 125 mg to about 750 mg, or about 125 mg to about 625 mg, or about 125 mg to about 500 mg, or about 125 mg to about 375 mg, or about 125 mg to about 250 mg, or about 250 mg to about 750 mg, or about 250 mg to about 625 mg, or about 250 mg to about 500 mg, or about 250 mg to about 375 mg, or about 375 mg to about 750 mg, or about 375 mg to about 625 mg, or about 375 mg to about 500 mg, or about 500 mg to about 750 mg, or about 500 mg to about 625 mg, or about 625 mg to about 750 mg of Compound 1.

In some embodiments, the present invention provides a method of treating, stabilizing or lessening the severity or progression of an arthritic condition, for example, rheumatoid arthritis, the method comprising administering to a patient in need thereof a therapeutically effective amount of Compound 1, wherein the therapeutically effective amount is a total daily dose selected from about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, a total daily dose of Compound 1 is administered as a single dose. In some embodiments, a total daily dose of Compound 1 is administered as two, three or four doses in one day, wherein each dose is identical. In some embodiments, a total daily dose of Compound 1 is administered as two, three or four doses in one day, wherein at least one dose is different from another dose. When more than one dose is administered in one day, the doses are independently selected from about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, a pharmaceutically acceptable composition comprising Compound 1 is administered twice daily. In some embodiments, a pharmaceutically acceptable composition comprising Compound 1 is administered “BID”. In some embodiments, a pharmaceutically acceptable composition comprising Compound 1 is administered in two different doses, wherein the first administered dose differs from the second administered dose.

In some embodiments, a pharmaceutically acceptable composition comprising Compound 1 is administered three times a day. In some embodiments, a pharmaceutically acceptable composition comprising Compound 1 is administered “TID”. In some embodiments, a pharmaceutically acceptable composition comprising Compound 1 is administered in three different doses, wherein at least one of the administered doses differs from another administered dose. In some embodiments, a pharmaceutically acceptable composition comprising Compound 1 is administered four times a day. In some embodiments, a pharmaceutically acceptable composition comprising Compound 1 is administered “QID”. In some embodiments, a pharmaceutically acceptable composition comprising Compound 1 is administered in four different doses, wherein at least one of the administered doses differs from another administered dose.

In some embodiments, a total daily dose of Compound 1 is administered once daily (QD), wherein the dose is selected from 75 mg, 100 mg, 125 mg, 250 mg, 375 mg, 500 mg, 625 mg or 750 mg. In some embodiments, Compound 1 is administered 125 mg QD. In some embodiments, a total daily dose of Compound 1 is administered twice daily, wherein each dose is independently selected from 75 mg, 100 mg, 125 mg or 250 mg. In some embodiments, Compound 1 is administered 125 mg BID. In some embodiments, Compound 1 is administered 250 mg BID.

In some embodiments, provided methods comprise administering to a patient a total daily dose of 125 mg. In some embodiments, provided methods comprise administering to a patient a total daily dose of 250 mg. In some embodiments, provided methods comprise administering to a patient a total daily dose of 375 mg. In some embodiments, provided methods comprise administering to a patient a total daily dose of 500 mg.

In some embodiments, a total daily dose of 375 mg of Compound 1 is administered to a patient twice a day, wherein the first administered dose differs from the second administered dose. In some such embodiments, a total daily dose of 375 mg of Compound 1 is administered as one 250 mg dose and one 125 mg dose.

In some embodiments, a total daily dose of 375 mg of Compound 1 can be administered as a 250 mg dose administered at a given timepoint (for example, in the morning) and a 125 mg dose administered at a later timepoint (for example, in the evening).

In some embodiments, a total daily dose of 375 mg of Compound 1 is administered according to the following dosing schedule:

(i) about 250 mg administered in the morning; and

(ii) about 125 mg administered in the evening.

In some such embodiments, the two doses are administered at least 4 hours apart. In some embodiments, the two doses are administered at least 8 hours apart. In some embodiments, the two doses are administered at least 12 hours apart. In some such embodiments, one dose (for example, 250 mg) is administered in the morning and the second dose (for example, 125 mg) is administered in the evening.

In some embodiments, a therapeutically effective amount of Compound 1 is administered over a period of 28 consecutive days (“a 28-day cycle”). In some embodiments, a therapeutically effective amount of Compound 1 is administered for two, three, four, five or six 28-day cycles. In some embodiments, a therapeutically effective amount of Compound 1 is administered for seven, eight, nine, ten, eleven, twelve or more 28-day cycles. In some embodiments, a pharmaceutically acceptable composition comprising Compound 1 is administered for at least thirteen, at least fourteen, at least fifteen, at least sixteen, at least seventeen, at least eighteen, at least nineteen or at least twenty 28-day cycles. In some embodiments, a therapeutically effective amount of Compound 1 is administered to a patient for the duration of the patient's life.

In some embodiments, two adjacent 28-day cycles may be separated by a rest period. Such a rest period may be one, two, three, four, five, six, seven or more days during which the patient is not administered a unit dose of Compound 1. In a preferred embodiment, two adjacent 28-day cycles are continuous.

In some embodiments, the total daily dose is selected from about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some embodiments, a total daily dose of Compound 1 is administered as a single dose. In some embodiments, a total daily dose of Compound 1 is administered as two, three or four doses in one day, wherein each dose is identical. In some embodiments, a total daily dose of Compound 1 is administered as two, three or four doses in one day, wherein at least one dose is different from another dose. When more than one dose is administered in one day, the doses are independently selected from about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg or about 750 mg.

In some such embodiments, a total daily dose of 375 mg of Compound 1 is administered to a patient twice a day, wherein the first administered dose differs from the second administered dose. In some embodiments, a total daily dose of 375 mg of Compound 1 comprises a 250 mg dose and a 125 mg dose, wherein each of the 250 mg dose and the 125 mg dose are administered at different times during one day.

### IV. Formulations Comprising Compound 1

As described above, provided methods comprise administering to a patient in need thereof a pharmaceutically acceptable composition comprising Compound 1, wherein the pharmaceutically acceptable composition is an oral dosage form. In some embodiments, the pharmaceutically acceptable composition is formulated as a capsule.

In certain embodiments, provided methods comprise administering to a patient in need thereof a pharmaceutically acceptable composition which comprises Compound 1, and one or more pharmaceutically acceptable excipients, such as, for example, binders, film coatings, diluents, disintegrants, wetting agents, lubricants and adsorbents, or combinations thereof. One skilled in the art will readily appreciate that the category under which a particular component is listed is not intended to be limiting; in some cases a particular component might appropriately fit in more than one category. Also, as will be appreciated, the same component can sometimes perform different functions, or can perform more than one function, in the context of a particular formulation, for example depending upon the amount of the ingredient and/or the presence of other ingredients and/or active compound(s). In some embodiments, the pharmaceutically acceptable composition is a blended powder.

### i. Binders and Diluents

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel®), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, malitol and xylitol, or a combination thereof. In some embodiments, a provided composition comprises a binder of microcrystalline cellulose and/or lactose monohydrate.

### ii. Disintegrants

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

### iii. Wetting Agents

Wetting agents, also referred to as bioavailability enhancers, are well known in the art and typically facilitate drug release and absorption by enhancing the solubility of poorly-soluble drugs. Representative wetting agents include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the wetting agent is a poloxamer. In some such embodiments, the poloxamer is poloxamer 407. In some embodiments, compositions for use in the present invention comprise from about 1% to about 30% by weight of wetting agent, based upon total weight of the blended powder.

### iv. Lubricants

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

### v. Adsorbents

Pharmaceutical compositions of the present invention may further comprise one or more adsorbents. Representative adsorbents include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% adsorbent, based upon total weight of given formulation.

### vi. N-(3-(5-fluoro-2-(4-(2-methoxyethoxyl)phenylamino)pyrimidin-4-ylamino)phenyl)acrylamide besylate

As described above, the present invention provides a method of treating an arthritic condition, the method comprising administering to a patient in need thereof a pharmaceutically acceptable composition comprising Compound 1. Thus, in some embodiments, provided methods comprise administering to a patient in need thereof a besylate salt of Compound 1.

In some embodiments, provided methods comprise administering to a patient in need thereof a pharmaceutically acceptable composition comprising from about 5% to about 60% of the besylate salt of Compound 1, based upon total weight of the formulation. In some embodiments, provided methods comprise administering to a patient in need thereof a pharmaceutically acceptable composition comprising from about 5% to about 15% or about 7% to about 15% or about 7% to about 10% or about 9% to about 12% of the besylate salt of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of the besylate salt of Compound 1, based upon total weight of the formulation. In certain embodiments, provided methods comprise administering to a patient in need thereof a pharmaceutically acceptable composition comprising from about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of the besylate salt of Compound 1, based upon total weight of given composition or formulation.

In some such embodiments, provided methods comprise administering to a patient in need thereof a pharmaceutical composition comprising a unit dose of Compound 1, wherein Compound 1 is in the form of a besylate salt. In some such embodiments, the unit dose is an amount sufficient to provide about 25 mg, about 50 mg, about 75 mg, about 100 mg, about 125 mg, about 150 mg, about 175 mg, about 200 mg, about 225 mg or about 250 mg of the free base of Compound 1. In some embodiments, the pharmaceutical composition comprising the besylate salt of Compound 1 is a solid oral dosage form.

In some embodiments, the present invention provides a method of treating, stabilizing or lessening the severity or progression of an arthritic condition, wherein said method comprises administering to a patient in need thereof the besylate salt of Compound 1 or a pharmaceutically acceptable composition thereof.

In some embodiments, the present invention provides a method of treating, stabilizing or lessening the severity or progression of rheumatoid arthritis, wherein said method comprises administering to a patient in need thereof the besylate salt of Compound 1 or a pharmaceutically acceptable composition thereof.

In some embodiments, the present invention provides a method of treating, stabilizing or lessening the severity or progression of an arthritic condition, for example, rheumatoid arthritis, the method comprising administering to a patient in need thereof a pharmaceutical composition comprising the besylate salt of Compound 1, wherein the amount of besylate salt is sufficient to deliver about 75 mg, about 100 mg, about 125 mg, about 250 mg, about 375 mg, about 500 mg, about 625 mg or about 750 mg of the free base of Compound 1. In some such embodiments, the pharmaceutical composition further comprises one or more pharmaceutically acceptable excipients selected from binders, film coating, diluents, disintegrants, wetting agents, lubricants and adsorbents. In some such embodiments, the pharmaceutical composition comprises one or more pharmaceutically acceptable excipients selected from microcrystalline cellulose, lactose monohydrate, sodium starch, poloxamer 407, fumed silica and magnesium stearate. In some embodiments, the pharmaceutical composition is selected from those in Table 1:

TABLE 1
Pharmaceutical Formulations Comprising Compound 1

| | Amount per | Amount per |
|---|---|---|
| Component | 25 mg Capsule | 125 mg Capsule |
| Capsule shell | 1, size 0 | 1, size 0 |
| | white capsule | white capsule |
| N-(3-(5-fluoro-2-(4-(2- | 34.97 mg | 174.30 mg |
| methoxyethoxy)phenylamino) | (25 mg free base) | (125 mg free base) |
| pyrimidin-4-ylamino)phenyl) | | |
| acrylamide besylate (API) | | |
| Microcrystalline cellulose | 186.03 mg | 101.68 mg |
| Lactose monohydrate | 32.50 mg | 41.50 mg |
| Sodium starch glycolate | 32.50 mg | 41.50 mg |
| Poloxamer 407 | 32.50 mg | 41.50 mg |
| Fumed silica | 3.25 mg | 4.15 mg |
| Magnesium stearate | 3.25 mg^{†} | 10.38 mg^{‡} |

^{†}0.5% (1.625 mg) intragranular; 0.5% (1.625 mg) extragranular.
^{‡}2.0% (8.30 mg) intragranular; 0.5% (2.08 mg) extragranular.

### V. Process for Preparing Pharmaceutical Compositions

Dry Blend Process:

Milled N-(3-(5-fluoro-2-(4-(2-methoxyethoxyl)phenylamino)pyrimidin-4-ylamino)phenyl)acrylamide besylate, milled microcrystalline cellulose, milled sodium starch glycolate, milled lactose monohydrate, milled poloxamer 407, and sieved fumed silica are weighed and mechanically blended. An intragranular portion of sieved magnesium stearate (0.5% or 2.0%, per Table 1) is added to the blender and the formulation blended. This blended formulation is then roller compacted, milled, and then blended. The remainder or extragranular portion of the magnesium stearate (0.5%, per Table 1) is added and the final formulation is blended. Capsules are either mechanically filled or manually filled via the flood fill method.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1

### General Protocols

B Lymphocyte Isolation for in vitro signaling, proliferation, and activation: Human naïve, primary B cells (CD19+, IgD+) were isolated from anti-coagulated whole blood by density centrifugation through Histopaque®-1077 and PBMC isolation. PBMCs were subject to red blood cell lysis using Red Blood Cell Lysis Buffer (Boston Bioproducts) followed by incubation with MACS reagent (130-091-150) and negative selection over a MACS column to obtain naïve primary B cells with >85% purity.

Immunoblot Analysis: Cells were incubated in serum-free RPMI media for 1-1.5 h. Isolated human B cells were incubated with Compound 1 at a final concentration of 0.001, 0.01, 0.1 and 1 μM. Ramos cells were incubated with increasing concentrations of Compound 1 (0.3 nM-3 μM). Cells were then incubated in the presence of Compound 1 for 1 h at 37° C. Following incubation, cells were centrifuged and resuspended in 100 μL of serum-free RPMI and BCR was stimulated with addition of 5 μg/mL α-human IgM. Samples were centrifuged, washed in PBS, and lysed in 100 μL of Cell Extraction buffer (Invitrogen FNN0011) plus 1:10 (v:v) PhosStop® Phosphatase Inhibitor and 1:10 (v:v) Complete® Protease Inhibitor (Roche 11836145001). Antibodies used for immunoblot analysis include P-PLCγ2 (CST 3872), PLCγ2 (CST 3871), Syk (CST 2712), P-Syk (CST 2710), Btk (BD 611116), P-Btk (Epitomics 2207), and Tubulin (Sigma T6199). Membranes were scanned on a LiCor Odyssey Scanner using infrared fluorescence detection.

Spleen Homogenization: Spleens were harvested from mice, frozen immediately in liquid nitrogen and stored at −80° C. To generate spleen lysates, each spleen was sliced in half and lysed using a Precellys 24 Bead Homogenizer in 500 μL of BioRad Bio-Plex Lysis Buffer plus protease inhibitors. Supernatant was transferred to a fresh microfuge tube and stored frozen at −80° C. until analysis.

B Lymphocyte Proliferation (³H-Thymidine Incorporation): A suspension of resting purified naïve human B-cells isolated by negative selection (MACS reagent 130-091-150) in RPMI was prepared at 0.4-0.5×10⁶ cells per mL. Cells were mixed together with α-human IgM (final concentration of 5 μg/mL in each well) and vehicle (DMSO) or Compound 1 (final concentrations of 0.01, 0.1, 1.0, 10.0, 100.0, or 1000 nM per well) and seeded in a 96-well plate. Cells were incubated for 56 h in a humidified incubator maintained at 37° C. and 5% CO₂. ³H-thymidine was added (final concentration of 1 μCi in each well) and cells were incubated overnight, harvested, and measured for ³H incorporation. Experiments were performed in triplicate.

### Example 2

Compound 1 is a potent, selective inhibitor of Btk. Compound 1 was rationally designed to possess high affinity for the ATP binding pocket and to form a specific covalent bond with cysteine 481 in Btk, a poorly conserved amino acid among kinases. In biochemical assays, Compound 1 is a potent inhibitor of Btk kinase activity (IC₅₀ₐₚₚₐᵣₑₙₜ<0.5 nM, k_{inact}/K_{I}=7.69×10⁴ M⁻¹ s⁻¹) and is highly selective. Because biochemical kinase assays may overestimate the potency of small molecule kinase inhibitors due to high ATP concentrations found in the cellular environment, cell activity for several of these closely related kinase family members was assessed.

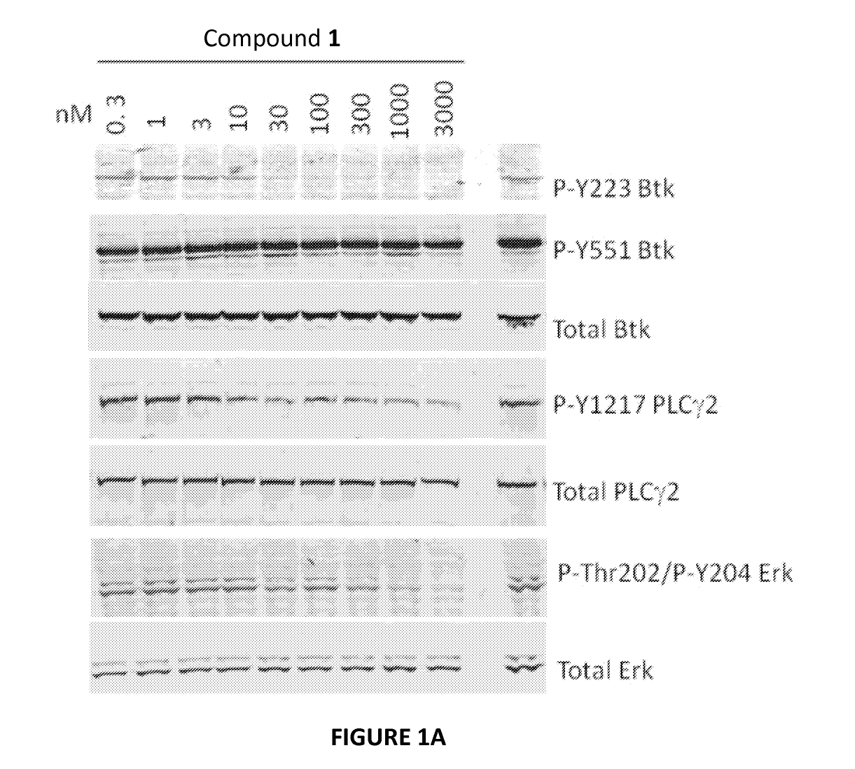

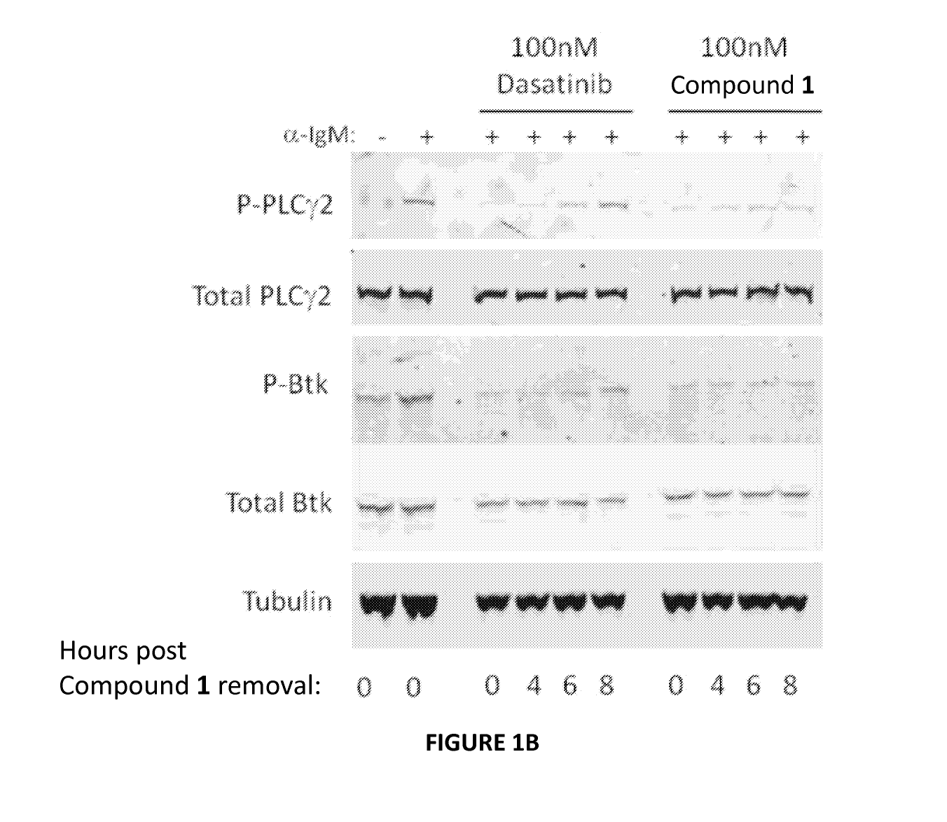

To ascertain the prolonged duration of action of Compound 1 after α-human IgM stimulation, cells were stimulated with 5 μg/ml α-IgM at 0, 4, 6, or 8 hours after compound removal and Btk substrate phosphorylation was measured by immunoblot. Btk remains inhibited up to 8 hours after treatment with the covalent modifier Compound 1, whereas Btk activity returns quickly after treatment with the reversible inhibitor, dasatinib (FIG. 1B).

Compound 1 demonstrated a high degree of selectivity against kinases with a cysteine in a homologous position as Cys 481 in Btk (EGFR, Itk, Jak3). To demonstrate specific inhibition of Btk in cells, Compound 1 was evaluated in Ramos cells which express an intact BCR signaling pathway that is activated robustly by addition of anti-IgM. Compound 1 potently inhibited Btk autophosphorylation on Tyr223 (EC₅₀=8 nM, FIG. 1A), phosphorylation of the Btk substrate, PLCγ2, as well as activation of the downstream kinase, Erk, all previously shown to be sensitive to Btk inhibition (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080; Di Paolo et al., “Specific Btk inhibition suppresses B cell- and myeloid cell-mediated arthritis,” Nat. Chem. Biol. 2011, 7:41-50). Importantly, while Compound 1 inhibited autophosphorylation of Btk, it had no effect on the phosphorylation of Btk on Tyr551, a site phosphorylated by Lyn and Syk and required for Btk activation (Afar et al., “Regulation of Btk by Src family tyrosine kinases,” Mol. Cell Biol. 1996, 16:3465-347). These data demonstrate Compound 1 is selective for Btk and does not inhibit the Src-family kinases upstream of Btk in the BCR signaling pathway (FIG. 1A).

Consistent with its covalent mechanism of action, Compound 1 provided prolonged inhibition of kinase activity hours after the drug was removed from cells. In contrast to reversible inhibition with the potent Btk inhibitor Dasatinib (Hantschel et al., “The Btk tyrosine kinase is a major target of the Bcr-Abl inhibitor dasatinib,” Proc. Natl. Acad. Sci. U.S.A 2007, 104:13283-13288), for which kinase activity had almost completely returned 6 hours after drug removal, recovery of Btk activity following a one hour exposure to Compound 1 continued to be suppressed ˜8 hours in drug-free media (FIG. 1B). This prolonged period of Btk inhibition correlated well with Btk protein turnover assayed in the presence of the protein synthesis inhibitor cycloheximide. These experiments indicated that existing cellular Btk was degraded slowly (36% reduction of protein in 8 hours and 63% reduction at 17 hours). Since Btk exposed to Compound 1 is irreversibly bound and inhibited, the return of Btk-dependent signaling relies on the appearance of new Btk protein as a result of protein synthesis in an Compound 1-free environment.

### Example 3

Quantitative Analysis of Btk Occupancy. The covalent mechanism of action of Compound 1 has enabled design of a companion PD assay that directly quantifies covalent bonding to Btk protein after drug exposure. A probe (Compound 2) was developed consisting of a covalent Btk inhibitor chemically linked to biotin:

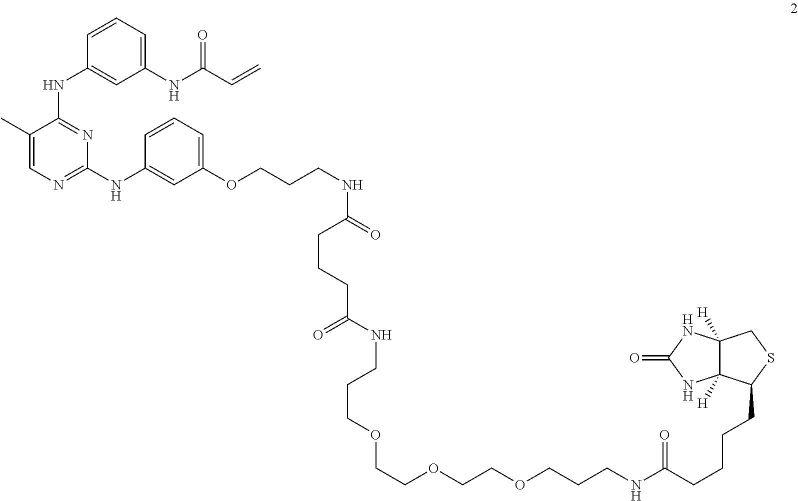 [SMILES: C=CC(=O)Nc1cccc(Nc2nc(Nc3cccc(OCCCNC(=O)CCCC(=O)NCCCOCCOCCOCCCNC(=O)CCCC[C@@H]4SC[C@@H]5NC(=O)N[C@@H]54)c3)ncc2C)c1]

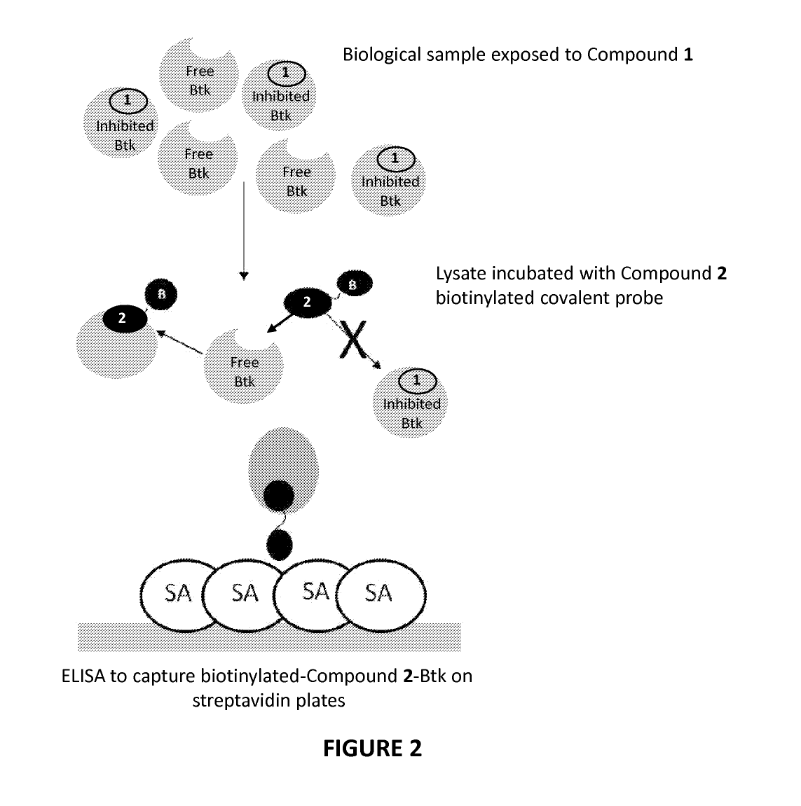

Compound 2 retains inhibitory activity against Btk (IC₅₀ₐₚₚ=0.5 nM) as well as the ability to form a covalent bond with Btk and has demonstrated selectivity against the structurally related kinase EGFR (IC₅₀ₐₚₚ>25 nM), and upstream Src-family kinases including Syk (IC₅₀ₐₚₚ>1000 nM) and Lyn (IC₅₀ₐₚₚ>3500 nM). Moreover, the specificity of the Btk target occupancy ELISA derives from the use of a detection monoclonal antibody that selectively recognizes Btk immobilized on the streptavidin substrate by the covalent probe and, therefore, this assay measures only Btk bound to the covalent probe (FIG. 2). By building a standard curve with known amounts of recombinant Btk protein bound to Compound 2, the amount of Btk in any sample can be precisely quantitated. Used in a competition assay, this probe detected free, uninhibited Btk and was excluded from interaction with Btk previously bonded by Compound 1 (FIG. 2). Results from this analysis can be reported in absolute values, such as the pg free Btk/ug total protein or in relative terms by normalization to control samples not exposed to inhibitor.

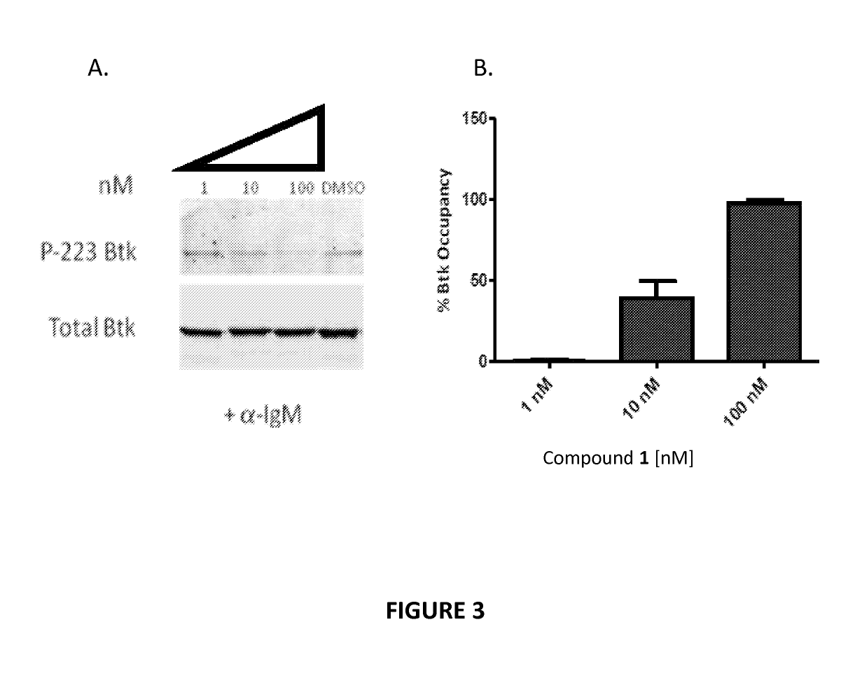

Btk Target Site Occupancy ELISA: An ELISA method for the detection of free uninhibited Btk in mouse, rat, dog, monkey, and human lysates was developed at Celgene Avilomics Research and a validation of this method in human B cell lysate was performed by a CLIA Certified laboratory (Cambridge Biomedical Laboratories, Boston, Mass.) The parameters that were assessed included: accuracy, linearity, dilution, precision (intra and inter-assay), stability, reference range, freeze-thaw cycles, reportable range, specificity, sensitivity, and carryover. All specifications for linearity, precision (intra- and inter-assay), accuracy, and carryover defined in the validation protocol were met. Samples were stable at −80° C. for 5 weeks and the reportable range of the Btk ELISA was 12.5 to 12,800 pg of free Btk. Cell lysates or spleen homogenates were incubated with Compound 2 (final concentration 1 μM) in a PBS, 0.05% Tween-20, 1% BSA solution for 1 h at room temperature. Standards and samples were transferred to a streptavidin-coated 96-well ELISA plate and mixed while shaking for 1 h at room temperature. The α-Btk antibody (BD 611116, 1:1000 dilution in PBS+0.05% Tween-20+0.5% BSA) was then incubated for 1 h at room temperature. After wash, goat α-mouse-HRP (1:5000 dilution in PBS+0.05% Tween-20+0.5% BSA) was added and incubated for 1 h at room temperature. The ELISA was developed with addition of tetramethyl benzidine (TMB) followed by Stop Solution and read at OD 450 nm. A representative immunoblot is presented in FIG. 3A. Btk occupancy (%) is presented in FIG. 3B. Uninhibited Btk detected from samples was normalized to μg total protein as determined by BCA protein analysis (Pierce Cat. 23225).

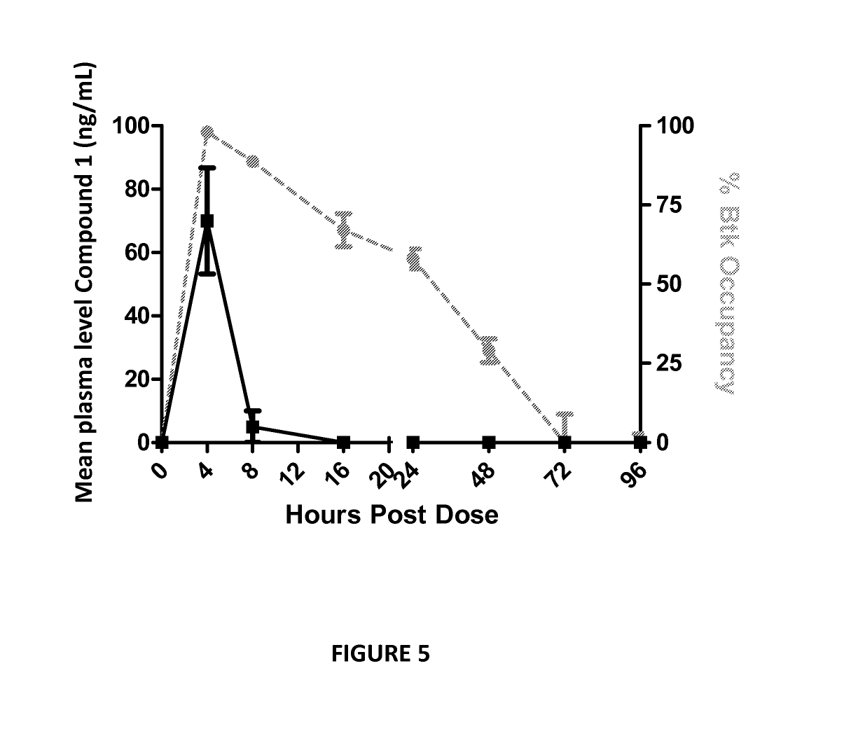

Btk Protein Resynthesis in Mice. Mice were treated orally once with 50 mg/kg Compound 1 to inhibit all Btk protein. At 4, 8, 16, 24, 48, 72, and 96 hours after treatment, spleens and serum were harvested (n=6 mice/timepoint). Spleens were homogenized and assayed on the covalent probe ELISA for Btk target site occupancy and compound concentration in serum was measured. Btk protein recovered to 50% pre-dose levels 24-48 hours after Compound 1 administration. Pharmacokinetic analysis of compound concentration in plasma at each timepoint demonstrated compound was undetectable in 5/6 mice by the 8 hour timepoint. Plotting the mean plasma level of Compound 1 in mice vs. the mean % Btk occupancy (both ±SEM) demonstrates Compound 1 action on Btk protein persists for >12 hours after circulating compound has disappeared (FIG. 5).

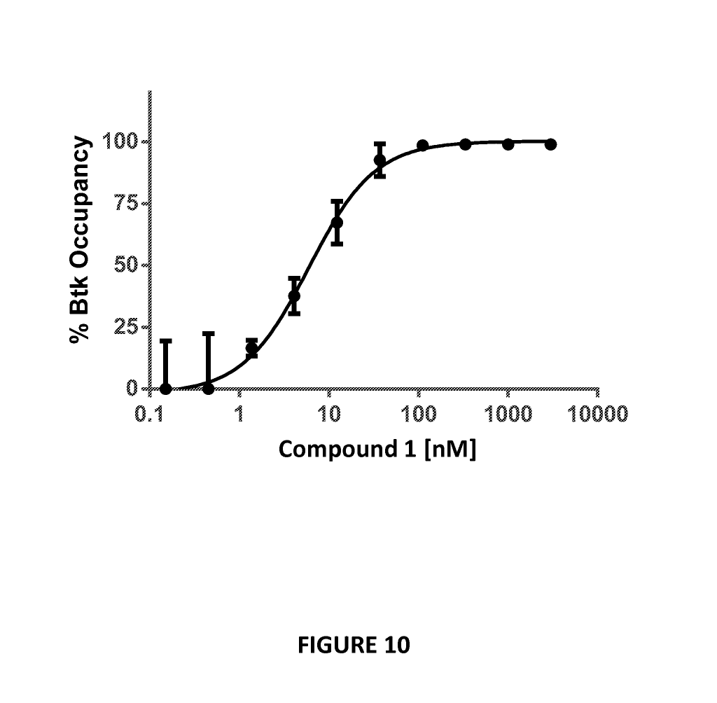

Results. In Ramos cells exposed to a range of Compound 1 concentrations, the amount of Btk captured by the probe was compared to untreated samples and the extent of Btk bonded was demonstrated to be proportional to drug concentration. Importantly, the degree of Btk covalently bonded by Compound 1, herein referred to as Btk occupancy, correlated with inhibition of Btk kinase activity. Extensive analysis has revealed that the EC₅₀ of Btk occupancy from a Compound 1 dose-response in Ramos cells (EC₅₀=6 nM) correlated directly with the cellular EC₅₀ of Btk kinase inhibition with Compound 1 (EC₅₀=8 nM). Such results demonstrate a near stoichiometric relationship. Furthermore, the concentration at which Compound 1 inhibited 90% of Btk activity in Ramos cells was 35 nM while the concentration of Compound 1 required for 90% occupancy of Btk was 39 nM, supporting a direct stoichiometric correlation between target occupancy and inhibition of Btk activity (FIG. 10).

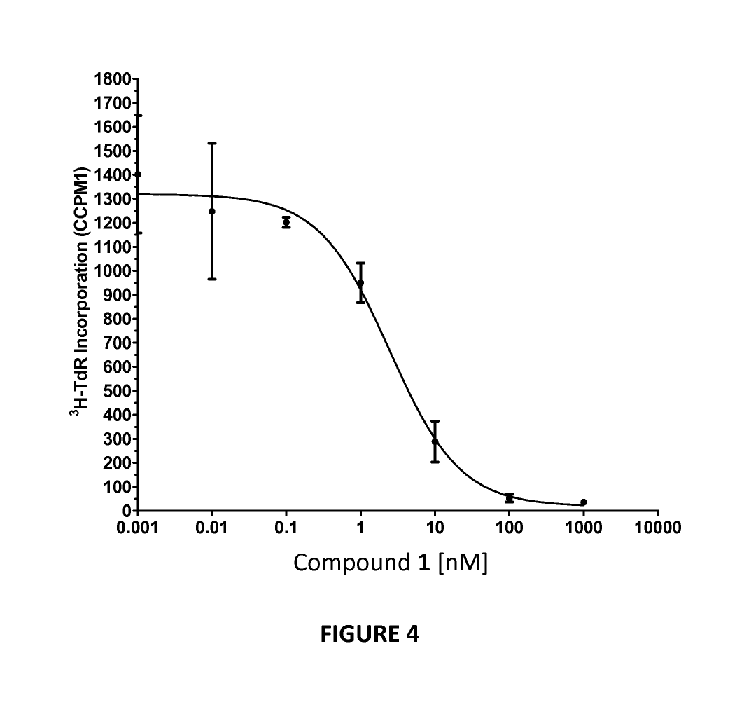

This correlative relationship was also demonstrated in freshly isolated human primary naïve B cells ex vivo. In naïve human B cells, the kinase activity of Btk was inhibited 42% at 10 nM, a concentration that produced 37% Btk occupancy (FIGS. 3A and 3B). Importantly, kinase inhibition and occupancy also reflected efficacy in B cell functional assays such as B cell proliferation (EC₅₀ 3 nM; FIG. 4) and activation as determined by inhibition of upregulation of the activation marker, CD69, in response to stimulation by anti-IgM. These data demonstrate a strong quantitative relationship among Compound 1 concentration, extent of Btk enzyme inhibition, and level of Btk occupancy. Therefore, measurement of Btk occupancy can serve as a robust surrogate measurement of Btk kinase inhibition that correlates with inhibition of BCR signaling and its functional consequences.

As described above, once covalently bound by Compound 1, an individual Btk protein is permanently silenced. Therefore, the return of activity must depend on new Btk protein synthesis. Determination of Btk protein re-synthesis rates in mice in vivo was enabled by maximally inhibiting Btk with a single dose of Compound 1 and then monitoring the return of Btk in spleen lysates over time with the covalent probe. Mouse spleens were collected at several time points after a single oral dose of 50 mg/kg Compound 1, a dose level projected to achieve complete Btk engagement, and assayed with the covalent probe to track emergence of new Btk protein. New Btk protein was detected at low levels 8 hours after compound administration, and achieved 43% of pre-dose Btk protein levels at 24 hours and 71% of pre-dose levels 48 hours after drug administration (FIG. 5). Importantly, pharmacokinetic (PK) analysis of mouse plasma from this experiment indicated circulating Compound 1 was absent in 5 of 6 animals by the 8 hour time point (data not shown). These data provide precise determination of the extent and duration of covalent inhibition of Btk protein in mice.

Given a long protein half-life, highly restricted expression pattern, and the presence of a poorly conserved cysteine in the ATP binding pocket, Btk represents an excellent target for selective covalent inhibition. The long protein half-life of Btk, shown previously to be >12 hours in human primary B cells (Saffran et al., “A Point Mutation in the SH2 Domain of Bruton's Tyrosine Kinase in Atypical X-Linked Agammaglobulinemia,” N. Engl. J. Med. 330, 1488-1491. 1994), provides for prolonged duration of drug action that extends well beyond the time frame of covalent drug exposure. The uncommon cysteine targeted by Compound 1 confers the opportunity for selective inhibition of Btk, as only 10 of the approximately 500 human kinases share placement of the cysteine in a homologous location within the ATP binding pocket. Importantly however, even in kinases sharing this cysteine, selectivity can be achieved with thoughtful drug design. Finally, since Btk is readily accessible in peripheral blood cells, target engagement can be measured easily by covalent probe ELISA.

### Example 4

### Relationship of Btk Occupancy and Efficacy of Compound 1 Besylate in the Collagen-Induced Arthritis (CIA) Model of Arthritis

The collagen-induced arthritis (CIA) model has been shown previously to respond to both B cell modulating therapies as well as direct Btk inhibition (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080; Chang et al., “The Bruton tyrosine kinase inhibitor PCI-32765 ameliorates autoimmune arthritis by inhibition of multiple effector cells,” Arthritis Res. Ther. 2011, 13:R115; Di Paolo et al., “Specific Btk inhibition suppresses B cell- and myeloid cell-mediated arthritis,” Nat. Chem. Biol. 2011, 7:41-50; Pine et al., “Inflammation and bone erosion are suppressed in models of rheumatoid arthritis following treatment with a novel Syk inhibitor,” Clin. Immunol. 2007, 124:244-257; Liu et al., “Therapeutic effects of TACI-Ig on collagen-induced arthritis by regulating T and B lymphocytes function in DBA/1 mice,” Eur. J. Pharmacol. 2011, 654:304-314). Oral efficacy of Compound 1 besylate in an established CIA model in mice was measured.

Established Collagen Induced Arthritis Model: Experiments were carried out at Bolder Biopath, Boulder, Colo. All experiments were carried out in compliance with regulations of the Institutional Animal Care and Use Committee and were conducted in accordance with principles and procedures dictated by the highest standards of humane animal care. Dba1 mice were injected at the base of the tail with 150 μL of Freund's Complete Adjuvant (Sigma) containing bovine type II collagen (Elastin Products, Owensville, Mo.) (2 mg/ml) on day 0 and again on day 21. On study days 25-27, onset of arthritis occurred, and mice were randomized into treatment groups (10/treatment group, 4/group for normal). Four treatment groups received Compound 1 besylate (3, 10 or 30 mg/kg) and one treatment group received dexamethasone (0.2 mg/kg). Randomization into each group was done after swelling was obviously established in at least one paw, and attempts were made to assure approximately equal mean scores across the groups at the time of enrollment. Treatment was initiated after enrollment of the dexamethasone and three of the Compound 1 besylate treatment groups (3, 10 and 30 mg/kg PO). Treatment continued daily (QD at 24 h intervals) through arthritis day 14. Treatment of the fourth Compound 1 besylate treatment group (30 mg/kg) began at peak disease (arthritis day 8) and continued daily (QD at 24 h intervals) through arthritis day 14. Clinical scores were assessed for each of the paws on study arthritis days 1-15 using the following scoring system: 0=normal, 1=one hind or fore paw joint affected or minimal diffuse erythema and swelling, 2=two hind or fore paw joints affected or mild diffuse erythema and swelling, 3=three hind or fore paw joints affected or moderate diffuse erythema and swelling, 4=marked diffuse erythema and swelling or =four digit joints affected, 5=severe diffuse erythema and severe swelling entire paw, unable to flex digits. Spleens and plasma were harvested 2 or 24 h after the last dose of Compound 1 besylate on arthritis day 14 and paws were removed and fixed in formalin for histopathological analysis.

Inflammatory Chemokine and Cytokine Analysis: At the termination of the CIA study, mice were sacrificed and blood was harvested via cardiac punch using EDTA as an anticoagulant. Following centrifugation, serum was diluted 1:4 with a mouse serum diluents kit (BioRad cat#171-305004). Levels of various mouse cytokines in plasma were quantified using Bioplex® Suspension Array System according to manufacturer's instruction (BioRad, mTNFa (171-G5023M), mIL-6 (171-G5007M), mKC (171-G5018M), mIFNg (171-G5017M), mIL-17 (Z60-00006Z2), mIL-1β (171-G5002M), mMCP-1 (171-G5019M).

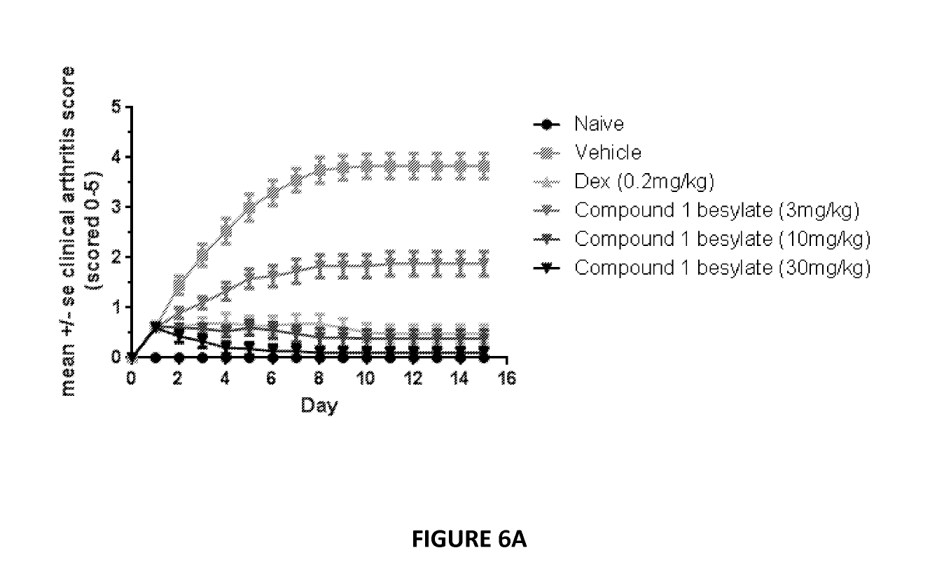

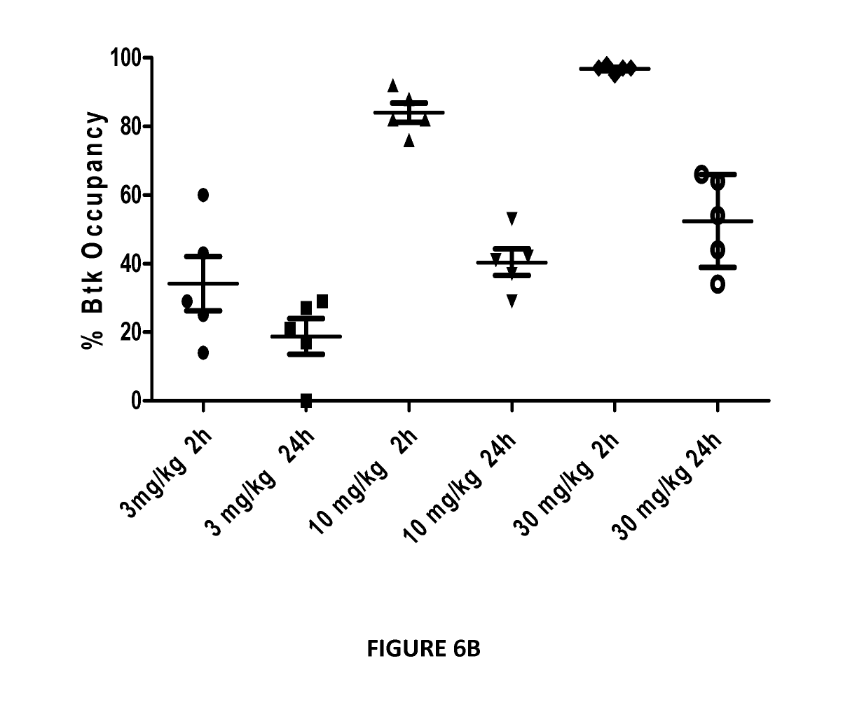

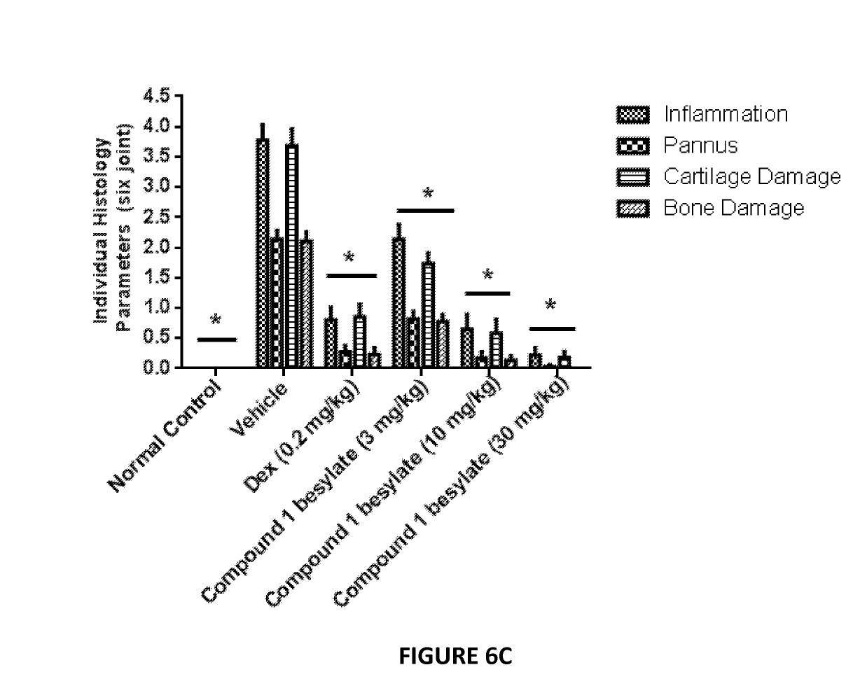

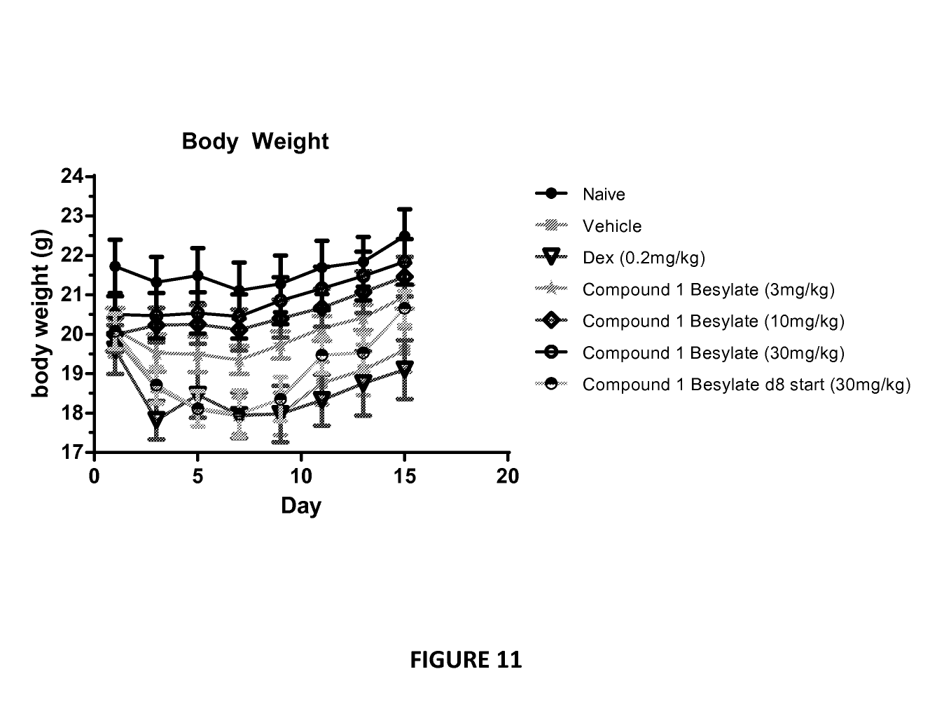

Compound 1 besylate demonstrated dose-dependent inhibition of the clinical signs of inflammatory disease was observed during the in-life portion of the model including reduction in joint and paw swelling and visible redness of the affected paws. Reduction of clinical signs of disease was measured at 95%, 85% and 50% for 30, 10 and 3 mg/kg respectively. (FIG. 6A). Moreover, all three dose levels of Compound 1 besylate prevented the loss in body weight typically associated with severity of disease observed in this model (FIG. 11). Compound 1 besylate administered at either 10 or 30 mg/kg was similar to dexamethasone control in inhibiting disease symptoms. Importantly, Compound 1 besylate also demonstrated significant effects on the generation of inflammatory chemokines and cytokines in this model including KC, IL-6, and TNFa. Such data are presented in Table 2, below.

TABLE 2
Effect of Compound 1 besylate on inflammatory
chemokines and cytokines

| | KC (pg/mL) | | IL-6 (pg/mL) | | TNF-a (pg/mL) | |
|---|---|---|---|---|---|---|
| | mean | sem | mean | sem | mean | sem |
| Naïve | 39.8 | 2.5 | 6.2 | 0.3 | 172.2 | 4.8 |
| Vehicle | 94.6 | 13.1 | 21.1 | 3.8 | 159.4 | 10.6 |
| Dexamethasone | 52.9 | 16.9 | 9.8* | 1.1 | 131.0 | 12.3 |
| 3 mg/kg Compound 1 | 62.0 | 11.5 | 15.4 | 2.5 | 160.2 | 12.0 |
| besylate | | | | | | |
| 10 mg/kg Compound 1 | 31.1* | 5.1 | 9.1* | 0.7 | 123.0* | 5.8 |
| besylate | | | | | | |
| 30 mg/lkg Compound 1 | 17.5* | 2.7 | 10.5* | 1.2 | 117.0* | 3.9 |
| besylate | | | | | | |

*p-value < 0.05 using Student's two-tailed t-test to vehicle

The precise mechanism for this protective effect is currently under investigation but suggests direct or indirect modulation of effect or cell function and may be independent of the role of Btk in B cells.

To demonstrate the relationship between inhibition of inflammatory activity and direct engagement of Compound 1 besylate with Btk, spleens collected either 2 or 24 hours after the last Compound 1 besylate dose were assayed for Btk occupancy. Occupancy in spleen lysates tracked closely with inhibition of the clinical signs of disease: 34% occupancy at 3 mg/kg at 2 hours correlated with 50% inhibition of disease, Btk occupancy of 84% was detected 2 hours after dosing with 10 (85% inhibition of disease) or 30 mg/kg (97% occupancy, 95% inhibition of disease) Compound 1 besylate. Consistent with Btk re-synthesis experiments described earlier, only 19% Btk occupancy remained 24 hours after the 3 mg dose whereas sustained occupancy of >40% at 24 hours was achieved with dose levels of 10 and 30 mg. This analysis demonstrated that once a day dosing at the higher doses resulted in continuous Compound 1 besylate-Btk engagement at levels greater than 40% and that this was sufficient for >85% inhibition of disease with therapeutic dosing of Compound 1 besylate (FIG. 6B). Morphologic and histopathologic analysis of 6 affected joints (4 paws, 2 knees) demonstrated a dose-dependent protection of joint damage including pannus formation, cartilage degradation, and bone erosion. The disease-modifying activity of Compound 1 besylate correlated with both Btk occupancy and the pronounced inhibition of the clinical inflammation characteristic of arthritis in this model (FIG. 6C). This correlation between Btk occupancy and inhibition of disease strongly suggests that selective inhibition of Btk provided the protective effect of Compound 1 besylate activity in this collagen-induced arthritis model.

Compound 1 besylate demonstrated therapeutic efficacy in a mouse CIA model, with 85% and 95% inhibition of disease observed at doses of 10 mg/kg/day and 30 mg/kg/day, respectively. This reduced severity of disease with Btk inhibition recapitulates the phenotype seen in xid mice, which harbor an inactivating mutation in the Btk gene and have a reduced incidence and severity of CIA disease induction (Mangla et al., “Pleiotropic consequences of Bruton tyrosine kinase deficiency in myeloid lineages lead to poor inflammatory responses,” Blood 2004, 104:1191-1197). These findings are also consistent with previously published results demonstrating that pharmacological inhibition of Btk reduced disease activity in autoimmune models (Honigberg et al., “The Bruton tyrosine kinase inhibitor PCI-32765 blocks B-cell activation and is efficacious in models of autoimmune disease and B-cell malignancy,” Proc. Natl. Acad. Sci. U.S.A 2010, 107:13075-13080; Chang et al., “The Bruton tyrosine kinase inhibitor PCI-32765 ameliorates autoimmune arthritis by inhibition of multiple effector cells,” Arthritis Res. Ther. 2011, 13:R115; Di Paolo et al., “Specific Btk inhibition suppresses B cell- and myeloid cell-mediated arthritis,” Nat. Chem. Biol. 2011, 7:41-50; Liu et al., “Antiarthritis effect of a novel Bruton's tyrosine kinase (BTK) inhibitor in rat collagen-induced arthritis and mechanism-based pharmacokinetic/pharmacodynamic modeling: relationships between inhibition of BTK phosphorylation and efficacy,” J. Pharmacol. Exp. Ther. 2011, 338:154-163). In addition to a full phenotypic response, once daily, oral dosing of 10 mg/kg Compound 1 besylate resulted in 84% Btk inhibition verified by Btk occupancy analysis assayed 2 hours after dose administration. Btk occupancy measured 24 hours after a 10 mg/kg dose suggested drug-free Btk protein was re-synthesized to approximately 60% of pre-dose levels. That sustained protection from the clinical signs of arthritis were provided at this dose suggests 100% Btk inhibition may not be required throughout a 24 hour time period and that intermittent inhibition of Btk may be sufficient for modulation of autoimmune disease in a clinical setting (Liu et al., “Antiarthritis effect of a novel Bruton's tyrosine kinase (BTK) inhibitor in rat collagen-induced arthritis and mechanism-based pharmacokinetic/pharmacodynamic modeling: relationships between inhibition of BTK phosphorylation and efficacy,” J. Pharmacol. Exp. Ther. 2011, 338:154-163).

### Example 5

Semi-Established Collagen Induced Arthritis (CIA) Arthritis Model. Experiments were carried out at Bolder Biopath, Boulder, Colo. All experiments were carried out in compliance with regulations of the Institutional Animal Care and Use Committee and were conducted in accordance with principles and procedures dictated by the highest standards of humane animal care. Dba1 mice were injected at the base of the tail with 150 μL of Freund's Complete Adjuvant (Sigma) containing bovine type II collagen (Elastin Products, Owensville, Mo.) (2 mg/ml) on day 0 and again on day 21. Mice were randomized into treatment groups on study day 21, and treatment was initiated. Treatment continued daily (3 mg/kg, 10 mg/kg or 30 mg/kg QD at 24 hour intervals) through day 33, and mice were terminated on day 33 (animals 1-6) or day 34 (animals 7-12). Onset of arthritis occurred between study days 21 and 34. Clinical scores were assessed for each of the paws on study days 21-34 using the following scoring system: 0=normal, 1=one hind or fore paw joint affected or minimal diffuse erythema and swelling, 2=two hind or fore paw joints affected or mild diffuse erythema and swelling, 3=three hind or fore paw joints affected or moderate diffuse erythema and swelling, 4=marked diffuse erythema and swelling or =four digit joints affected, 5=severe diffuse erythema and severe swelling entire paw, unable to flex digits. Spleens and plasma were harvested at necropsy (day 33 for animals 1-6 and day 34 for animals 7-12) and paws were removed and fixed in formalin for histopathological analysis.

Vehicle treated control mice had disease incidence of 83% on study day 33. Mice treated with 3 mg/kg Compound 1 had a disease incidence of 50% on study day 33. Mice treated with 10 mg/kg Compound 1 had a disease incidence of 17% on study day 33. Mice treated with 30 mg/kg Compound 1 had a disease incidence of 8% on study day 33. Mice treated with dexamethasone had a disease incidence of 0% on study day 33.

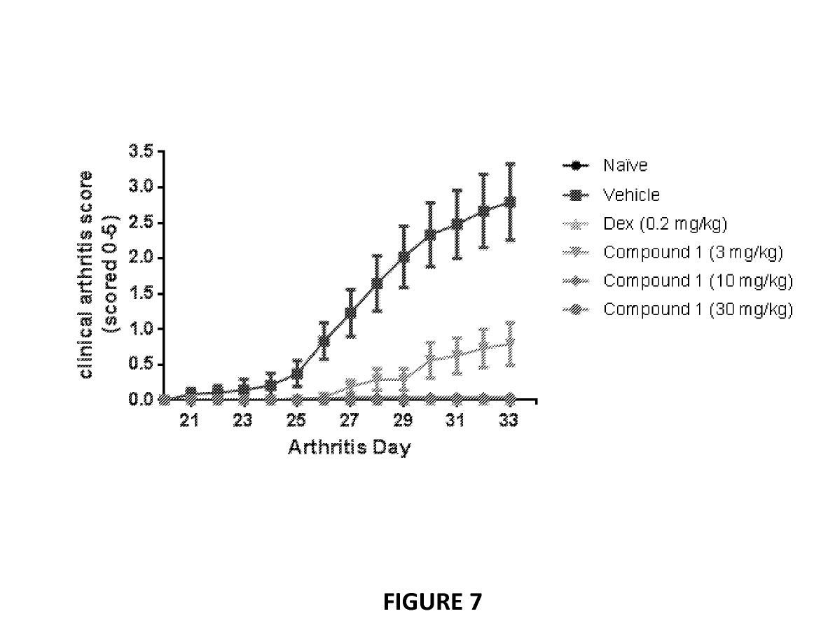

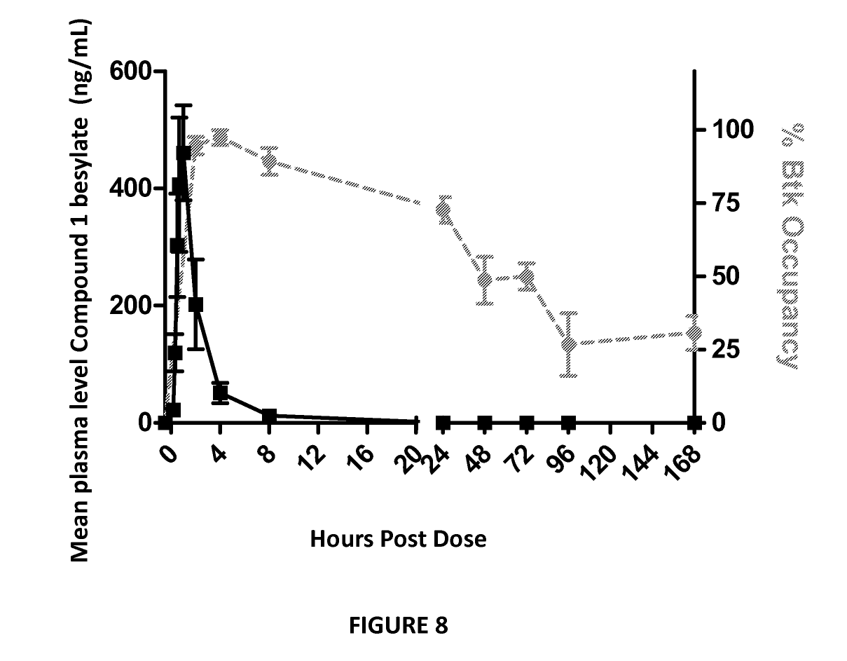

Daily treatment with Compound 1 besylate at 3 mg/kg, 10 mg/kg or 30 mg/kg had a significant beneficial effect on the clinical parameters associated with developing type II collagen arthritis in mice (FIG. 7). Moreover, all three dose levels of Compound 1 prevented the loss in body weight typically associated with severity of disease observed in this model. Compound 1 administered at either 10 or 30 mg/kg was similar to dexamethasone control in inhibiting disease symptoms.

### Example 6

### Human Clinical PK-PD Relationship with Compound 1 Besylate

Clinical Study: A double blind, placebo-controlled, ascending single dose, randomized study in normal healthy human volunteers was conducted at a single clinical research unit in accordance with Declaration of Helsinki principles. Informed consent statements were obtained from all subjects prior to inclusion in the study. Subjects were admitted to the unit 1 day before dosing and discharged 96 h after dosing. Six subjects were administered a single oral dose of 2 mg/kg Compound 1 besylate, monitored for safety and evaluated for drug action by PK and PD analysis.

Isolation of Enriched B Lymphocyte Population from Human Healthy Volunteers: 21 mL of human whole blood was collected from each subject at each time point into BD® Vacutainer® CPT Cell Preparation Tubes containing sodium heparin. RosetteSep™ Human B Cell Enrichment Cocktail (Stem Cell Tech. 15024) was added to each CPT tube and centrifuged for 25 minutes at 1800×g at room temperature. Isolated cells were harvested into a clean 50 mL conical tube that was pooled by subject. Each enriched B cell suspension was centrifuged at 400×g for 15 minutes at room temperature. Cell suspensions were diluted in 1 mL of red blood cell lysis buffer for 3 minutes at room temperature. Cell pellets were lysed with 150 μL Bio-Rad Bio-Plex lysis buffer (Cat. #171-304012). The lysates were stored frozen at ≦−70° C. until Btk target site occupancy analysis by ELISA.

Compound 1 besylate demonstrated covalent bonding, prolonged, selective inhibition of Btk in vitro, and efficacy in preclinical models in vivo. In addition, there was a strong correlation between the concentration of Compound 1 besylate required for Btk occupancy, inhibition of BCR signaling, and consequent functions such as B cell proliferation. As part of a larger clinical study with Compound 1 besylate in healthy adult human volunteers, traditional pharmacokinetic analysis of plasma drug levels was paired with Btk occupancy analysis in a B cell enriched fraction from freshly isolated human blood to determine the PK-PD relationship of Compound 1 besylate following single oral administration in humans. After initial dose escalation, 2 mg/kg Compound 1 besylate was found to be optimal for analysis of this PK-PD relationship. 6 healthy adult subjects were administered a single oral dose of Compound 1 besylate (2.0 mg/kg) and sequential blood samples were isolated over time to determine the relationship between the plasma concentration of Compound 1 besylate and Btk occupancy in an enriched B cell population.

2 mg/kg Compound 1 besylate was rapidly absorbed in all subjects with peak plasma concentrations achieved within 30-120 minutes after dose administration and a mean measured maximum plasma concentration of 542 ng/mL (Cmax) was attained. Plasma concentrations declined to near or below the lower limit of detection (0.1 ng/mL) within 24 hours post dose with a median terminal elimination half-life of 1.9 hours. Plasma concentrations of Compound 1 besylate at 48 hours post dose were below the lower limit of quantification in all subjects.

Analysis of Btk occupancy was determined at each time point using the covalent probe ELISA. The absolute value of free Btk in lysates of enriched B cells isolated before Compound 1 besylate administration averaged 465±67 pg free Btk/ug total protein (mean±SEM) for the 6 subjects administered Compound 1 besylate at a dose of 2.0 mg/kg. Within 4 hours after dose administration, 5 of 6 subjects had greater than 98% Btk occupancy with the 6^{th} subject achieving 84% occupancy. Complete or near-complete Btk occupancy was sustained in all 6 subjects for between 8-24 hours post administration of Compound 1 besylate and this occurred at a time when plasma concentrations of Compound 1 besylate were low or approaching the limit of quantification. Thus, as in the experiment in mice described above, detection of free Btk over the ensuing 24-96 hour period post administration of Compound 1 besylate was a reflection of the re-synthesis rate of Btk by existing B cells plus the addition of any new B cells circulating in the periphery. Free Btk protein levels recovered towards 75% pre-dose values within 96 hours with a re-synthesis half-life of 48-72 hours and an average re-synthesis rate of 3.0 pg Btk/ug protein/hour. These data demonstrate an uncoupling of PD from PK and, similar to data generated in preclinical models, reveal that Compound 1 besylate action on Btk in human clinical trials was sustained for several hours after circulating drug levels declined to undetectable levels (FIG. 7). The generation of metabolites was not evaluated in this clinical study and, therefore, the potential contribution of active metabolites of Compound 1 besylate cannot be excluded. These translational studies demonstrate the capability to precisely determine the concentration of Compound 1 besylate required for complete inhibition of Btk in human subjects to inform subsequent drug development.

In freshly isolated primary human B lymphocytes, there was a close correlation between the concentration of Compound 1 besylate required to inhibit Btk signaling, B cell proliferation, and achieve Btk occupancy, suggesting a quantitative relationship among inhibition of Btk kinase activity, target occupancy, and functional assays in vitro. This direct correlation supports the use of Btk occupancy as a surrogate marker for inhibition of Btk activity. Importantly, this relationship between the inhibition achieved and the extent of Compound 1 besylate-target engagement measured by the covalent probe was maintained in vivo.

As expected by its covalent mechanism of action, the PK of Compound 1 besylate was dissociated from PD in vivo, a feature confirmed by analysis of Btk occupancy. Compound 1 besylate remained active on Btk for a prolonged duration in vivo after plasma drug levels had declined to undetectable levels. Recovery from Compound 1 besylate treatment occurred slowly in both mice and humans as new Btk protein was made. In mice, where Btk occupancy was measured in spleen homogenates, Btk protein was re-synthesized to 50% of pre-dose levels 24-48 hours after a single dose. This differs from the re-synthesis of Btk protein seen in humans which required 48-72 hours to recover to 50% of baseline protein levels. This may reflect a more rapid re-synthesis rate of Btk in mice. However, this may also reflect the fact that in mice, Btk return was measured in spleen lysates whereas Btk re-synthesis in humans was measured in isolated peripheral B cells. In both mouse and human studies, Compound 1 besylate-Btk engagement persisted well after circulating free drug had disappeared. In this way, covalent inhibitors allow a departure from the confines of traditional drug design; a pharmacokinetic profile that includes a long circulating half-life to ensure 24 hour target coverage is not necessary. Instead, a covalent inhibitor must achieve in vivo concentrations sufficient to engage all available molecular target only for a short period and then the re-synthesis rate of the target itself dictates the duration of drug action. Moreover, the level and length of drug action can be empirically determined by target occupancy measurements to rationally adjust dosing to a schedule that achieves certain occupancy characteristics.

Although there are other Btk inhibitors in the clinic (Harrison, “Trial watch: BTK inhibitor shows positive results in B cell malignancies,” Nat. Rev. Drug Discov. 2012, 11:96; Ponader et al., “The Bruton tyrosine kinase inhibitor PCI-32765 thwarts chronic lymphocytic leukemia cell survival and tissue homing in vitro and in vivo,” Blood 2012, 119:1182-1189; Alinari et al., “Novel targeted therapies for mantle cell lymphoma,” Oncotarget. 2012, 3:203-211), Compound 1 besylate is the first specific covalent Btk inhibitor in human clinical trials. Once daily oral dosing of Compound 1 besylate can achieve complete and sustained Btk occupancy, a surrogate for Btk inhibition. The covalent mechanism of action of Compound 1 besylate on its intended target, Btk, was confirmed and PD was assessed in this first-in-human trial. This represents a marked acceleration of clinical PD evaluation, typically not available until Phase 2 clinical testing. The rapid identification of doses providing Btk engagement provides an advantage in the design of subsequent human clinical trials and supports Phase 2 dose selection incorporating safety, tolerability, and on-target activity.

### Example 7

PGPS 10S-Induced Model of Chronic Arthritis. Rats were injected intraperitoneal with PG-PS 10S, 15 μg/g of animal body weight, to induce arthritis. Left and right hind limb lateral ankle widths of each rat were measured three times each prior to PG-PS 10S administration and on day 5. The first inflammatory response peak for both right and left ankles of PG-PS rats was reached on day 4. Rats were assigned to one of five treatment groups. Rats were treated with 3 mg/kg QD, 10 mg/kg QD, 30 mg/kg QD or 30 mg/kg QOD (every other day) Compound 1 on the 3^{rd} day post first peak (day 7) through day 22. Another group was treated with 0.3 mg/kg QD dexamethasone on the 3^{rd} day post first peak (day 7) through day 22.

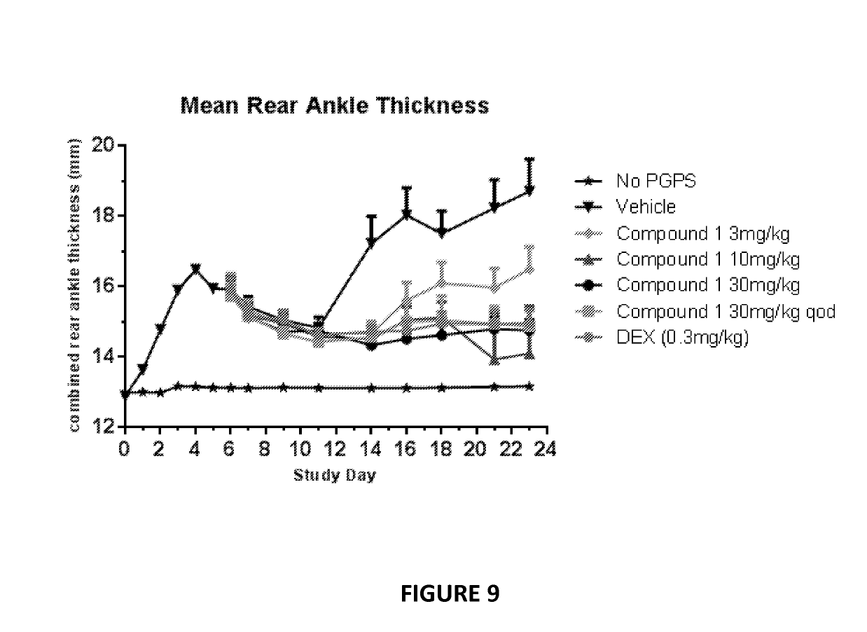

Following the first inflammatory response peak, ankle inflammation continued to decrease and reached a nadir on study day 11 for both left and right ankles of rats in most treatment groups including those in the vehicle group. The second inflammatory response phase (chronic phase) clearly started on study day 14 and reached a peak on study day 23. Right and left lateral ankle width measurement values obtained from rats treated with Compound 1 remained significantly lower compared to those rats in the diseased group (FIG. 9). PGPS 10S injected rats treated with the vehicle showed enlarged spleens that were surrounded with a fibrous-like connective tissue. Compound 1 reduced this effect of PGPS 10S on spleen enlargement in a dose-dependent manner. Compound 1 administration at 10 mg/kg QD, 30 mg/kg QD and 30 mg/kg QOD significantly prevented spleen enlargement when compared to the vehicle administration. Compound 1 inhibited PGPS 10S-induced arthritis in a dose-dependent manner.

### Example 8

### Clinical Study in Arthritic Human Subjects

The active pharmaceutical ingredient (API), Compound 1 besylate, is a chemically synthesized small molecule substituted pyrimidine developed as the benzenesulfonic acid salt and is a white to off-white crystalline powder. Compound 1 besylate is an oral, potent (IC₅₀<0.5 nM) and selective small molecule inhibitor of Btk. Compound 1 besylate exhibits solubility of approximately 0.16 mg/mL in water and a maximum aqueous solubility of 0.40 mg/mL at approximately pH 3.0. The solubility of Compound 1 besylate in ethanol is approximately 10 mg/mL. Compound 1 besylate exhibits no environmental instabilities (i.e. heat, acid, base) that require special handling.

The API was formulated into capsules containing the components and quantities listed in Table 2 to obtain the study drug.

TABLE 2
Compound 1 besylate capsules

| | Amount per |
|---|---|
| Component | 125 mg Capsule |
| Capsule shell | 1, size 0 |
| | white capsule |
| N-(3-(5-fluoro-2-(4-(2- | 174.30 mg |
| methoxyethoxy)phenylamino) | (125 mg free base) |
| pyrimidin-4-ylamino)phenyl) | |
| acrylamide besylate (API) | |
| Microcrystalline cellulose | 101.68 mg |
| Lactose monohydrate | 41.50 mg |
| Sodium starch glycolate | 41.50 mg |
| Poloxamer 407 | 41.50 mg |
| Fumed silica | 4.15 mg |
| Magnesium stearate | 10.38 mg^{‡} |

^{‡}2.0% (8.30 mg) intragranular; 0.5% (2.08 mg) extragranular.

Study Design. A multicenter, randomized, double-blind, placebo-controlled study will be initiated to determine the efficacy and safety of Compound 1 besylate on a stable background of MTX therapy in subjects with active rheumatoid arthritis. Approximately 80 subjects with active RA while on a stable dose of MTX background therapy, as defined by 2010 ACR/EULAR criteria (Table 3), will be randomized 1:1 (40 per treatment group) using an Interactive Voice Response System (IVRS) to receive either Compound 1 besylate or identically appearing placebo for 4 weeks. A total daily dose of 375 mg Compound 1 besylate will be administered orally (PO) as follows:

(i) a 250 mg dose administered in the morning; and

(ii) a 125 mg dose administered H.S. (at bedtime).

The 250 mg dose will be administered to patients under fasting conditions. The 125 mg dose will be administered to patients who have not eaten within 2 hours prior to administration.

TABLE 3
2010 ACR/EULAR Classification Criteria for Rheumatoid Arthritis⁺

| A. Joint Involvement | |
|---|---|
| 1 large joint | 0 |
| 2-10 large joints | 1 |
| 1-3 small joints (with or without involvement of large joints) | 2 |
| 4-10 small joints (with or without involvement of large joints) | 3 |
| >10 joints (at least 1 small joint) | 5 |
| B. Serology | |
| Negative RF and negative ACPA | 0 |
| Low-positive RF or low-positive ACPA | 2 |
| High-positive RF or high-positive ACPA | 3 |
| C. Acute-phase reactants | |
| Normal CRP and normal ESR | 0 |
| Abnormal CRP or abnormal ESR | 1 |
| D. Duration of symptoms | |
| <6 weeks | 0 |
| ≧6 weeks | 1 |

⁺scores from A-D are added to achieve a total score. A total score of 6/10 or greater is classified as definite RA.

Clinical efficacy for amelioration of signs and symptoms of RA (ACR20; ACR50; ACR70 response) will be determined at one endpoint of the study, or Week 4. The ACR20 response is defined as ≧20% improvement from baseline in the joint tenderness and joint swelling scores plus ≧20% improvement from baseline in 3 of the following 5 assessments: subject global assessment of disease activity (SGA); physician global assessment of disease activity (PGA); HAQ-DI score; subject assessment of pain; and the acute phase reactant (CRP). ACR50 and ACR70 response are ≧50% and ≧70% improvements from baseline, respectively. The ACR20 response is a well-validated and accepted standard for the evaluation of patients with RA and is commonly used as an endpoint in clinical trials. Clinical efficacy will be evaluated in the following major areas: signs and symptoms, physical function/health-related quality of life, and patient and physician reported outcomes. The laboratory results, adverse events, chest radiographs, vital signs, ECGs, ophthalmologic and physical exams will be monitored to evaluate safety.

Screening procedures will take place no more than 35 days prior to the start of study medication (Visit 2). Subjects meeting criteria for study entry will be randomly assigned to treatment with Compound 1 besylate plus their stable MTX therapy or placebo plus their stable MTX therapy at Visit 2. Clinic visits will occur at baseline (week 0), weeks 1, 2 and 4, as well as 28 days post completion of the double-blind treatment period to assess for safety and temporal onset of response. One dose reduction, from a 375 mg daily dose consisting of one 250 mg dose and one 125 mg dose PO to a single 125 mg PO administered in the morning under fasting conditions, will be allowed. Such dose reduction will be permitted under the following circumstances:
Any subject experiencing GI adverse events such as diarrhea, nausea and vomiting lasting for one day or more, at the discretion of the investigator, the subject may be treated with an intervention such as ondansetron, loperamide, etc. If the subject is unresponsive to the intervention (i.e. ondansetron, loperamide, etc) and lasting for one day or longer the subject must be dose-reduced. If the GI adverse event does not resolve after 3 days on the reduced dose, the subject should be discontinued from the investigational product
Any subject with elevated liver enzymes (AST or ALT>3×ULN to <8×ULN and total bilirubin≦2×ULN). Subjects with elevated liver enzymes should have a repeat test within 48 to 72 hours to determine if the dose reduction is required. If the retest confirms the transaminase elevation, the subject must be dose-reduced and followed until resolution or until the subject meets the criteria of a stopping rule
Any subject who meets the following rule for albumin and protein excretion (based on albumin/creatinine ratio and protein/creatinine ratio) and renal function (based on MDRD eGFR) must have their dose reduced (refer to Table 4):

TABLE 4

| | | |
|---|---|---|
| Albumin/creatinine | <30 | 31-300 |
| (mg/g) | | |
| Protein/creatinine | <50 | 51-500 |
| (mg/g) | | |
| MDRD eGFR | Continue on investigational | Decrease dose |
| ≧90 mL/min/1.73 m² | product; no dose reduction | from 375 mg PO |
| | required | daily to 125 mg |
| | | PO daily |
| ≧60-<90 ml/min/ | Decrease dose from 375 mg | Discontinue |
| 1.73 m² | PO daily to 125 mg PO daily | treatment |

The study population will consist of female and male subjects between 18 and 80 years of age (inclusive) at the time of signing the informed consent document (ICD). Subjects must have a diagnosis of active RA (2010 American College of Rheumatology [ACR]/European League Against Rheumatism [EULAR] Classification Criteria for Rheumatoid Arthritis) of at least 6 months duration despite treatment with adequate stable doses of MTX (7.5 to 25 mg/week oral or parenteral). Subjects must have been treated with MTX for a minimum of 4 months prior to randomization and on a stable dose of MTX for at least 12 weeks prior to randomization.

Active disease is defined as ≧6 swollen joints out of 66 swollen joint counts and ≧6 tender joints out of 68 tender joint counts at randomization, positive for rheumatoid factor (RF) plus at least two of the following laboratory measures:
hsCRP≧10 mg/L
Erythrocyte sedimentation rate (ESR)>28 mm after the first 1 hour
Positive for anti-cyclic citrullinated peptide antibodies (anti-CCP)

To provide supportive therapy to subjects randomized to placebo, all subjects will be required to take MTX as background therapy at the stable dose they have been taking for the preceding 4 months prior to randomization and remain on a stable dose (between 7.5 and 25 mg/week PO or parenteral) for at least 12 weeks prior to randomization through Day 28/Week 4. In addition, subjects are permitted to take the following medications as long as they remain on a stable dose that is maintained through Day 28/Week 4:
NSAIDs
Hydroxychloroquine or chloroquine
Sulfasalazine
Low-dose oral corticosteroids (prednisone≦10 mg/day or equivalent)

To assess for potential immune effects, the following safety measures have been incorporated; laboratory testing for serum immunoglobulin levels (IgM, IgG, and IgA) and lymphocyte subsets (CD3⁺, CD4⁺, CD8⁺, CD16⁺/56⁺, CD45, CD19+, CD20+, CD20−/CD27 Bright, CD38, and CD69). Subjects will also be screened for chronic viral hepatitis infections (hepatitis B and C).

Pharmacokinetic and PD biomarker measures will be assessed the extent of exposure and to explore the relationship between clinical efficacy, modulation of cytokines and other disease-related proteins and other effects (such as laboratory parameters). The analyses will include the following:
Btk occupancy
Btk signaling pathway markers (Btk, PLCγ2, and ERK phosphorylation)
B cell subsets (CD19, CD27, CD38, IgM, IgD) and activation markers (CD69) ex vivo analysis by flow cytometry
RA proteins (Vector DA Assay™)
   Adhesion molecules: vascular cell adhesion molecule 1 (VCAM-1)
   Growth factors: epidermal growth factor (EGF) and vascular endothelial growth factor A (VEGF-A)
   Cytokine-related proteins: interleukin 6 (IL-6) and tumor necrosis factor receptor, type 1(TNF-RI)
   Matrix metalloproteinases: MMP-1 and MMP-3
   Skeletal-related proteins: YKL-40
   Bone remodeling hormones: Leptin and Resistin
   Acute-phase proteins: serum amyloid (SAA) and hsCRP
Other RA biomarkers including but not limited to TNF-α, IL-8, haptoglobin, Regulated on Activation, Normal T cell Expressed and Secreted (RANTES), chemokine ligand 3 (CCL3) and chemokine ligand 4 (CCL4)
Markers of bone and cartilage catabolism, including but not limited to collagen type 1 C-telopeptides (CTX-I), collagen type 1 cross-linked N-telopeptide (NTX), and cartilage oligomeric matrix peptide (COMP)

### Example 9

Non-Obese Diabetic Mouse (NOD) Model of Sjogren's Syndrome. Male Non Obese Diabetic (NOD) mice from the KWS BioTest NOD colony and male BALB/c mice were randomly allocated to experimental groups and allowed to acclimatise for one week prior to the beginning of the study. From six weeks of age, animals were treated according to the administration schedule in Table 5, below. Each group consisted of n=12 animals. The administration volume was 15 ml/kg from 6-12 weeks of age. The administration volume was then decreased to 10 ml/kg from 13-20 weeks of age. The vehicle was distilled water pH 3.0-3.5 for Group 7. The vehicle was 0.5% methylcellulose and 0.25% Tween® 80 for all other groups.

TABLE 5
Sjogren's Syndrome Treatment Groups

| | | Dose | | | |
|---|---|---|---|---|---|
| | | Level | Administration | | Disease |
| Group | Treatment | (mg/kg) | Frequency | Route | Induction |
| 1 | Control | n/a | n/a | n/a | n/a |
| 2 | Vehicle | n/a | SID, Day 0-End | p.o. | Spontaneous |
| 3 | Compound 1 | 10 | SID, Day 0-End | p.o. | |
| | besylate | | | | |
| 4 | Compound 1 | 30 | SID, Day 0-End | p.o. | |
| | besylate | | | | |
| 5 | Compound 1 | 100 | SID, Day 0-End | p.o. | |
| | besylate | | | | |
| 6 | Compound 1 | 50 | BID, Day 0-End | p.o. | |
| | besylate | | | | |
| 7 | Compound 1 | n/a* | Ad libitum, Day | p.o. | |
| | besylate | | 0- End | | |

n/a: not applicable
SID (semel in die): once daily
BID (bis in die): twice daily
p.o. (per os): oral gavage
*solution at 0.22 mg/ml corresponding to 0.16 mg/ml of active compound

Also from six weeks of age, animals were monitored every other week for clinical signs of Sjogren's syndrome to include dacryoadenitis (inflammation of the lachrymal glands) and sialoadenitis (inflammation of the submandibular glands). Inflammation of the lachrymal glands and inflammation of the submandibular glands result in a loss of function. Lachrymal gland secretions were quantified by the cotton thread assay. Submandibular gland secretions were quantified by the pilocarpine-induced salivation assay.

At six weeks, ten weeks and twenty weeks of age, non-terminal blood samples were collected and processed to isolate serum. The serum samples were stored for anti-M3R antibody analysis.

At twenty weeks of age, terminal blood samples were collected 4 hours after the last treatment (half of the animals) or 24 hours after the last treatment (half of the animals) in K2-EDTA-coated tubes and processed to isolate plasma. The plasma samples were stored. Also at twenty weeks of age, spleens were dissected out 4 hours after the last treatment (half of the animals) or 24 hours after the last treatment (half of the animals), snap frozen and stored. At twenty weeks of age, the lachrymal glands and submandibular glands were dissected out and stored in tissue fixative for histopathology analysis.

Animals were weighed at the start of the study (Day 0) and once per week until termination. All animals were observed for signs of ill health throughout the study.

Glycaemia. From fourteen weeks of age, animals were monitored weekly for signs of hyperglycaemia. A blood sample was taken from a superficial vein and the blood glucose level was measured using Free Style Optium glucose test strips (Abbott) and a Boots glucose reader.

Measurement of lachrymal gland secretion. Lachrymal gland secretion was measured every other week from six weeks of age using a cotton thread test. Briefly, animals were anaesthetised and a fluorescein-stained cotton thread was inserted under the eye lid in the medial canthus region and held in place for two minutes. Results for each group were graphed and presented with SEM's.

Measurement of salivary gland secretion. Salivary gland secretion was measured every other week from six weeks of age. Briefly, animals were anesthetised and secretion was stimulated by intra-venous administration of pilocarpine hydrochloride (0.5 mg/kg). Saliva was collected for five minutes. The weight of saliva collected was then measured using digital scales. Results for each group were graphed and presented with SEM's.

Histopathology. Samples of lachrymal gland and salivary glands from all animals were fixed for Haematoxylin & Eosin histological analysis. Sections were inspected blind by a qualified histopathologist and scored for signs of pathology according to a semi-quantitative scoring system. This system is based on the mean number of inflammatory foci comprising 50 or more mononuclear inflammatory cells per objective field. The ratio of infiltrated to normal glandular tissue was expressed on a five point scale.

Analysis of anti-M3R Ab in serum. Samples of serum isolated on Weeks 6, 10 and 20 will be analysed by ELISA to determine the levels of anti-M3R Ab. Results will be presented as raw OD values and antibody quantity for each group and time-point will be graphed and presented with SEM's.

Spleen/Blood for plasma collection. At termination, groups were split in half, blood and spleen were collected at 4 hours or 24 hours post final dose (half of each group at each time-point). Blood was collected in K2-EDTA tubes, inverted a number of times to mix with the K2-EDTA and placed on ice until spun for plasma preparation. Plasma was prepared by spinning blood samples at 8000 rpm for 8 minutes. Spleens were snap-frozen over liquid nitrogen in cryovials. All samples were stored at −80° C.

Results. Three animals in Group 5 were found dead following the pilocarpine-induced salivation assay under isoflurane anaesthesia. Two animals, one in Group 3 and one in Group 5, did not recover from the isoflurane anaesthesis.

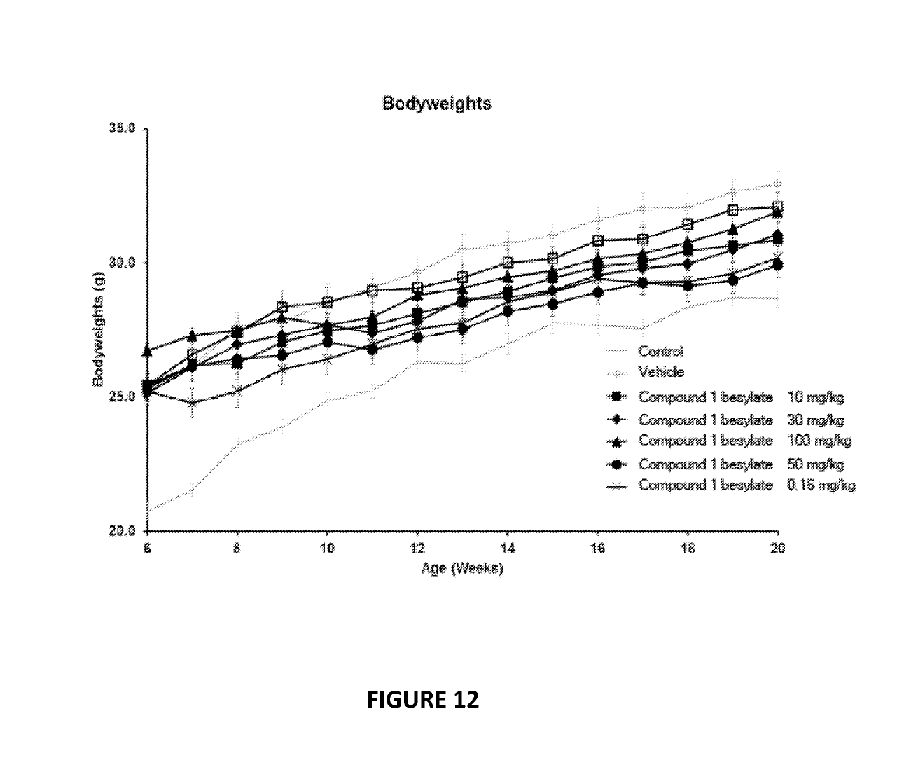

Bodyweight data, expressed in grams, were analysed by two-way ANOVA followed by Dunnett's post-test for multiple comparisons to the vehicle-treated group and presented as mean bodyweights for each group (FIG. 12).

The average bodyweight in the BALB/c (Control) group was significantly lower than the average bodyweight in age matched NOD mice treated with the vehicle. A highly significant difference was observed from 6 weeks of age until the end of the experiment at 20 weeks of age (p<0.0001). Compound 1 besylate administered at 10 mg/kg induced a significant decrease in bodyweight when compared to the vehicle-treated group at 13 weeks, 17 weeks, 19 weeks and 20 weeks of age (p<0.05). Compound 1 besylate administered at 30 mg/kg induced a significant decrease in bodyweight when compared to the vehicle-treated group from 14 weeks of age (corresponding to 8 weeks of treatment) until the end of the experiment at 20 weeks of age (p<0.05). Compound 1 besylate administered twice daily at 50 mg/kg induced a highly significant decrease in bodyweight when compared to the vehicle-treated group from 11 weeks of age (corresponding to 5 weeks of treatment) until the end of the experiment at 20 weeks of age (p<0.0001 to 0.01). Compound 1 besylate administered ad libitum at 0.16 mg/ml in the drinking water induced a highly significant decrease in bodyweight when compared to the vehicle-treated group at 8 weeks of age (p<0.01) and from 10 weeks of age (corresponding to 4 weeks of treatment) until the end of the experiment at 20 weeks of age (p<0.001 to 0.05).

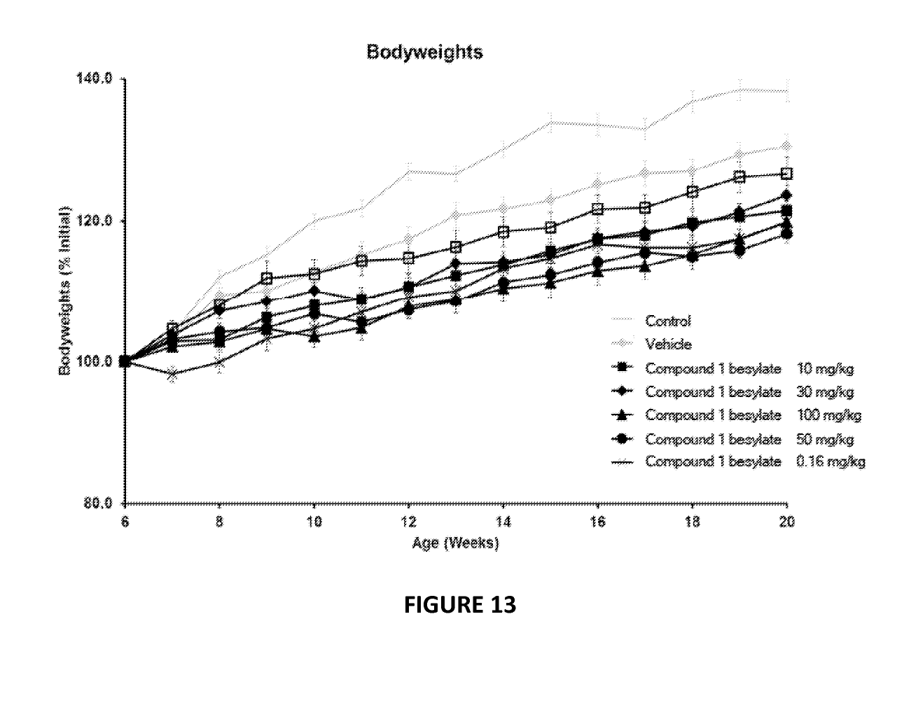

Bodyweight data, expressed as a percentage of the initial bodyweight, were analysed by two-way ANOVA followed by Dunnett's post-test for multiple comparisons to the vehicle-treated group and presented as mean percentage of the initial bodyweight ±SEM (FIG. 13).

Bodyweight increase in the BALB/c (Control) group was significantly higher than in the age matched group of vehicle-treated NOD mice from 10 weeks of age until the end of the experiment at 20 weeks of age (p<0.0001 to 0.05). Compound 1 besylate administered at 10 mg/kg induced a significant reduction in bodyweight gain when compared to the vehicle-treated group at 8 weeks of age and from 10 weeks of age (corresponding to 4 weeks of treatment) until 20 weeks of age. Compound 1 besylate administered at 30 mg/kg induced a significant reduction in bodyweight gain when compared to the vehicle-treated group from 11 weeks of age (corresponding to 5 weeks of treatment) until 20 weeks of age. Compound 1 besylate administered at 100 mg/kg induced a significant reduction in bodyweight gain when compared to the vehicle-treated group at 8 weeks of age and from 10 weeks of age (corresponding to 4 weeks of treatment) until 20 weeks of age. Compound 1 besylate administered twice daily at 50 mg/kg induced a significant reduction in bodyweight gain when compared to the vehicle-treated group from 10 weeks of age (corresponding to 4 weeks of treatment) until 20 weeks of age. Compound 1 besylate administered ad libitum at 0.16 mg/ml in the drinking water induced a highly significant reduction in bodyweight gain when compared to the vehicle-treated group from 8 weeks of age (corresponding to 2 weeks of treatment) until 20 weeks of age.

Glycaemia. Blood glucose was monitored once per week from 14 weeks of age. Animals with glycaemia equal to or greater than 11 mM on two consecutive weekly readings were considered diabetic. The incidence of diabetes in male NOD mice at 20 weeks of age was 5% (4/84 mice) (Table 6):

TABLE 6
Incidence of Diabetes

| | | Age (Weeks) | | |
|---|---|---|---|---|
| Group | | 18 | 19 | 20 |
| Control | | 0 | 0 | 0 |
| Vehicle | | 0 | 0 | 0 |
| Compound 1 | 10 mg/kg SID | 17% (2/12) | 17% (2/12) | 18% (2/11) |
| besylate | | | | |
| Compound 1 | 30 mg/kg SID | 0 | 0 | 0 |
| besylate | | | | |
| Compound 1 | 100 mg/kg SID | 0 | 0 | 0 |
| besylate | | | | |
| Compound 1 | 50 mg/kg BID | 0 | 0 | 0 |
| besylate | | | | |
| Compound 1 | 0.16 mg/kg | 8% (1/12) | 17% (2/12) | 17% (2/12) |
| besylate | ad libitum | | | |

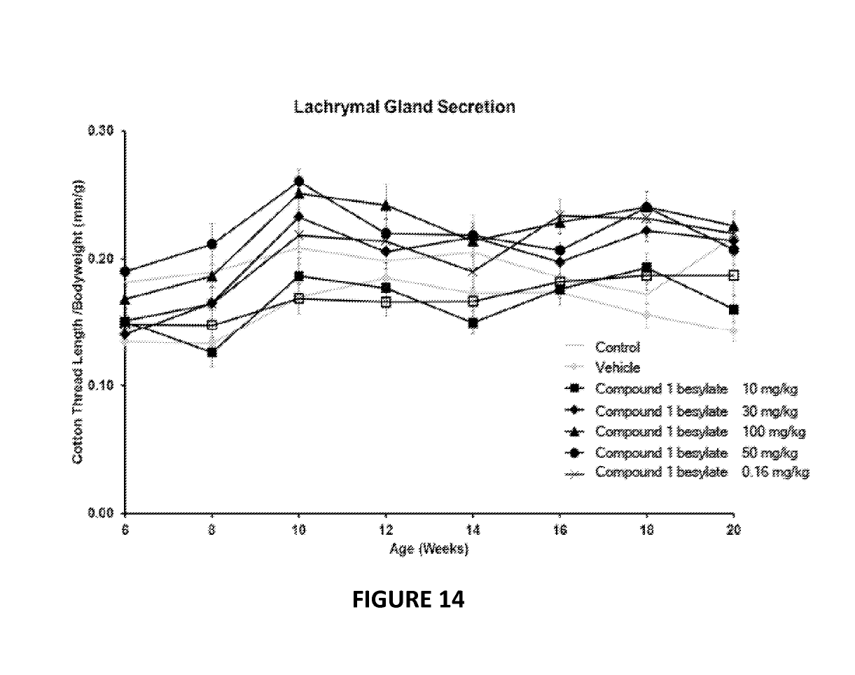

Lachrymal Gland Secretion. Lachrymal gland secretion was measured by the cotton thread test. Data are expressed as millimetres per gram bodyweight. Data from this readout are indicative of inflammatory changes in the lachrymal gland, but significant changes are typically only seen from weeks 18 onwards in NOD mice. Data were analysed by two-way ANOVA for repeated measures followed by Dunnett's post-test for multiple comparisons to the vehicle-treated group and presented as mean±SEM (FIG. 14).

Lachrymal gland secretion was lower in the vehicle-treated group of NOD mice than in the control group of BALB/c mice at all time-points tested. A highly significant difference was observed at 20 weeks of age (p<0.0001). Lachrymal gland secretion was significantly higher following Compound 1 besylate administered at 30 mg/kg when compared to the vehicle at 10 weeks (p<0.01), 18 weeks (p<0.01) and 20 weeks of age (P<0.001). Lachrymal gland secretion was significantly higher following Compound 1 besylate administered at 100 mg/kg when compared to the vehicle at 8 weeks (p<0.05), 10 weeks (p<0.0001), 12 weeks (p<0.01), 16 weeks (p<0.05), 18 weeks (p<0.0001) and 20 weeks of age (p<0.0001). Lachrymal gland secretion was significantly higher following Compound 1 besylate administered twice daily at 50 mg/kg when compared to the vehicle at 6 weeks (p<0.01), 8 weeks (p<0.0001), 10 weeks (p<0.0001), 18 weeks (p<0.0001) and 20 weeks of age (p<0.001). Lachrymal gland secretion was significantly higher following Compound 1 besylate administered ad libitum at 0.16 mg/ml in the drinking water when compared to the vehicle at 10 weeks (p<0.05), 16 weeks (p<0.01), 18 weeks (p<0.001) and 20 weeks of age (p<0.0001).

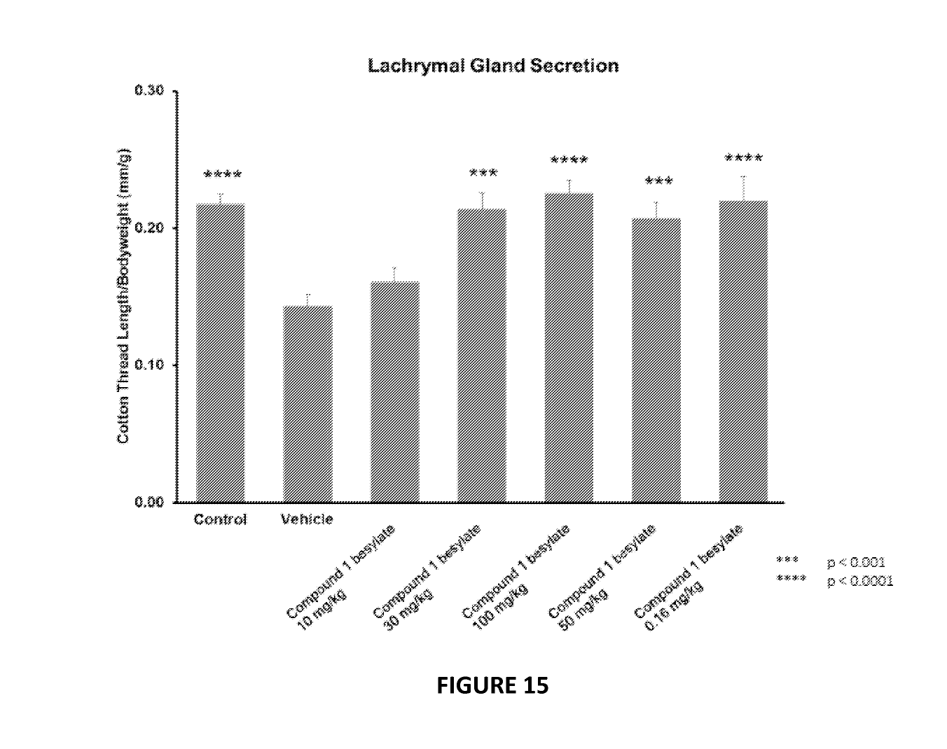

Data collected at 20 weeks of age were also analysed by one-way ANOVA followed by Dunnett's post-test for multiple comparisons to the vehicle-treated group (FIG. 15). An increase in lachrymal gland secretion in groups treated with Compound 1 besylate is indicative of a therapeutic benefit in treating at least one symptom of Sjogren's syndrome. Prior to treatment with Compound 1 besylate, a highly significant decrease in lachrymal secretion was observed in the vehicle-treated group of NOD mice when compared to the age-matched control group of BALB/c mice (p<0.0001). Lachrymal gland secretion was significantly higher following Compound 1 besylate administered at 30 mg/kg (p<0.001) and 100 mg/kg (p<0.0001), when compared to the vehicle. Lachrymal gland secretion was also significantly higher following Compound 1 besylate administered twice daily at 50 mg/kg (p<0.001) and following Compound 1 besylate administered ad libitum at 0.16 mg/ml in the drinking water (p<0.0001) when compared to the vehicle.

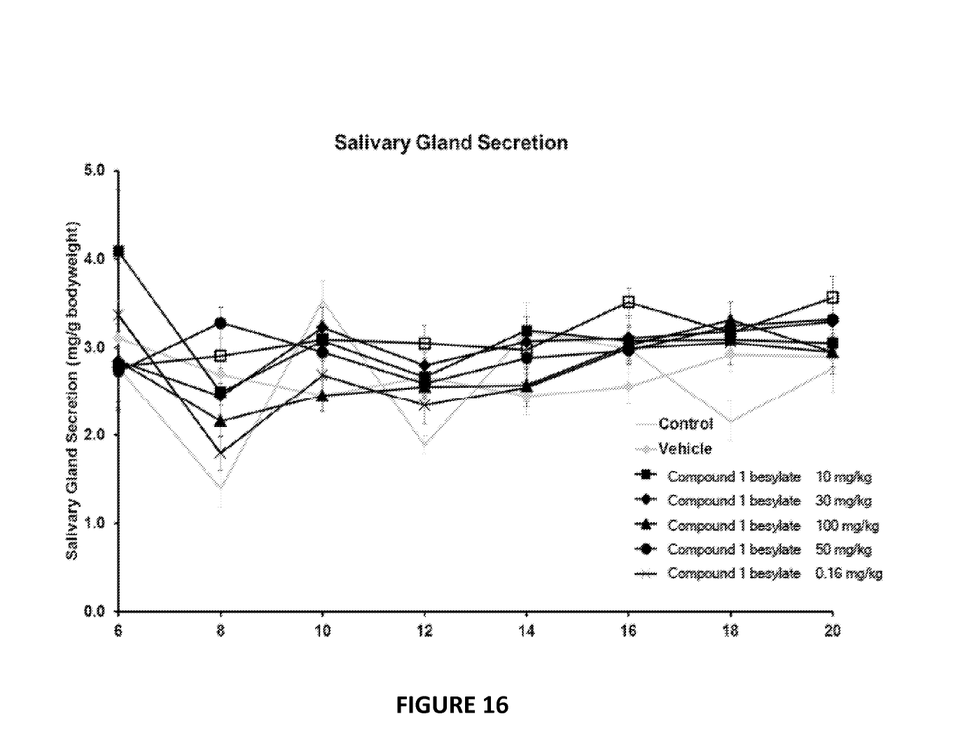

Salivary Gland Secretion. Salivary gland secretion was measured using the pilocarpine-induced salivation assay. Data are expressed as milligram of saliva per gram bodyweight. Data from this readout are indicative of inflammatory changes in the salivary glands, but significant changes are typically only seen from weeks 18 onwards in NOD mice. In male NOD mice, the salivary gland involvement in disease is much lower than lachrymal gland involvement. Data were analysed by two-way ANOVA followed by Dunnett's post-test for multiple comparisons to the vehicle-treated group (FIG. 16). No significant differences were observed in levels of salivary gland secretions between groups in this study.

Lachrymal Gland Histopathology. Lachrymal glands from Control BALB/c mice (n=12 mice) and vehicle-treated NOD mice (n=9 mice) were examined and scored, in order to validate the model. For each animal, one section from the right lachrymal gland and one section from the left lachrymal gland were prepared. Sections were examined and scored for two criteria:

(i) Focus Score. The mean number of inflammatory foci comprising ≧50 mononuclear inflammatory cells per ×10 objective field was counted. Five such fields were assessed (where possible) to derive this index and these are selected to contain the maximum number of such foci. Where the overall area of the tissue section was less than five fields, the number of ×10 objective fields was recorded and used to determine the focus score.
(ii) Area Score. The ratio of infiltrated to normal glandular tissue was expressed on a five point scale:
0: No infiltrates
1: 0-25% of the tissue section infiltrated
2: 26-50% of the tissue section infiltrated
3: 51-75% of the tissue section infiltrated
4: 76-100% of the tissue section infiltrated

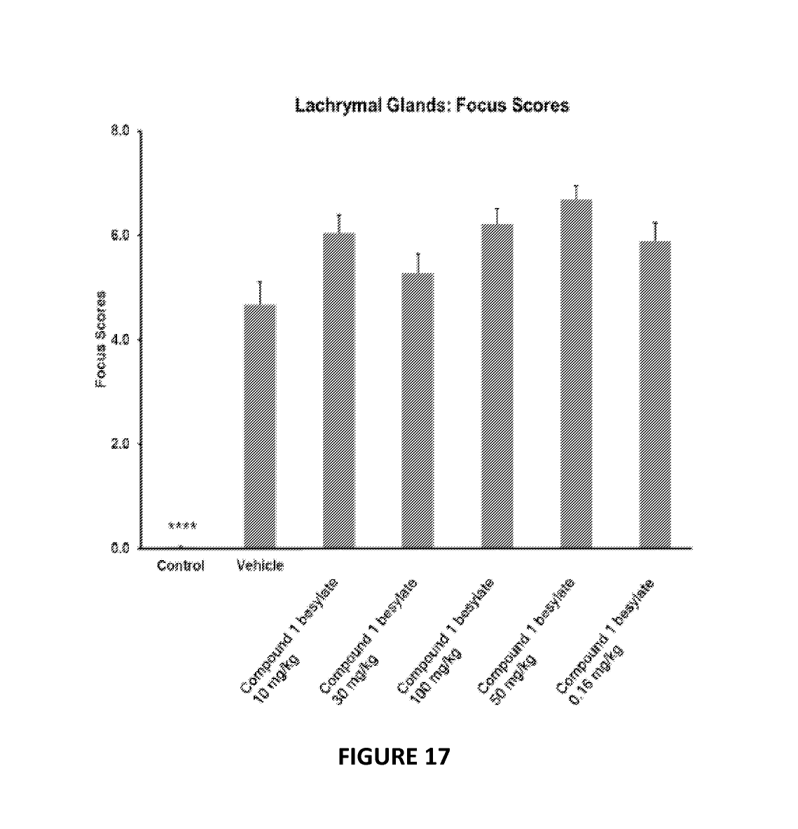

Focus Score data were analysed by Kruskal-Wallis test for non-parametric values followed by Dunn's post-test for multiple comparisons to the vehicle-treated group. A highly significant increase in the number of inflammatory foci was observed in the vehicle-treated group of NOD mice at 20 weeks of age when compared to age-matched control BALB/c mice (p<0.0001). Compound 1 besylate administration did not reduce the number of inflammatory foci when compared to the vehicle-treated group (FIG. 17).

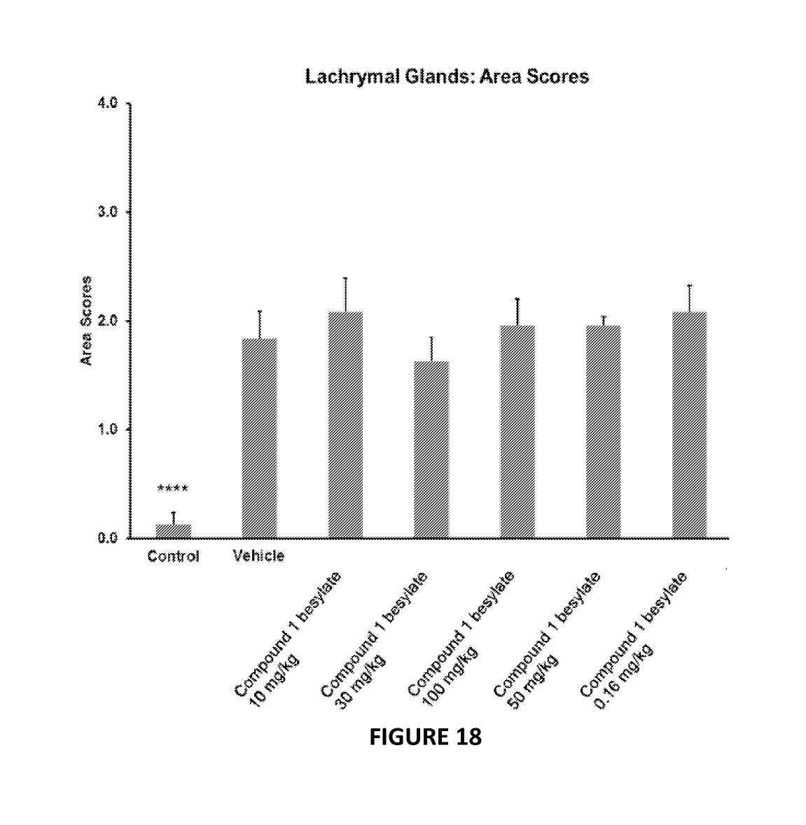

Area Score data were analysed by Kruskal-Wallis test for non-parametric values followed by Dunn's post-test for multiple comparisons to the vehicle-treated group. A highly significant increase in the proportion of infiltrated glandular tissue was observed in the vehicle-treated group of NOD mice at 20 weeks of age when compared to age-matched control BALB/c mice (p<0.0001). The percentage of tissue infiltrated in the vehicle-treated NOD mice was 45.8±6.4% when compared to 3.1±2.8% in the control group of BALB/c mice. Compound 1 besylate administration did not reduce the proportion of infiltrated glandular tissue when compared to the vehicle-treated group (FIG. 18).

Salivary Gland Histopathology. Salivary gland sections were prepared for each animal. Sections were examined and scored for two criteria: Focus Score and Area Score as outlined above.

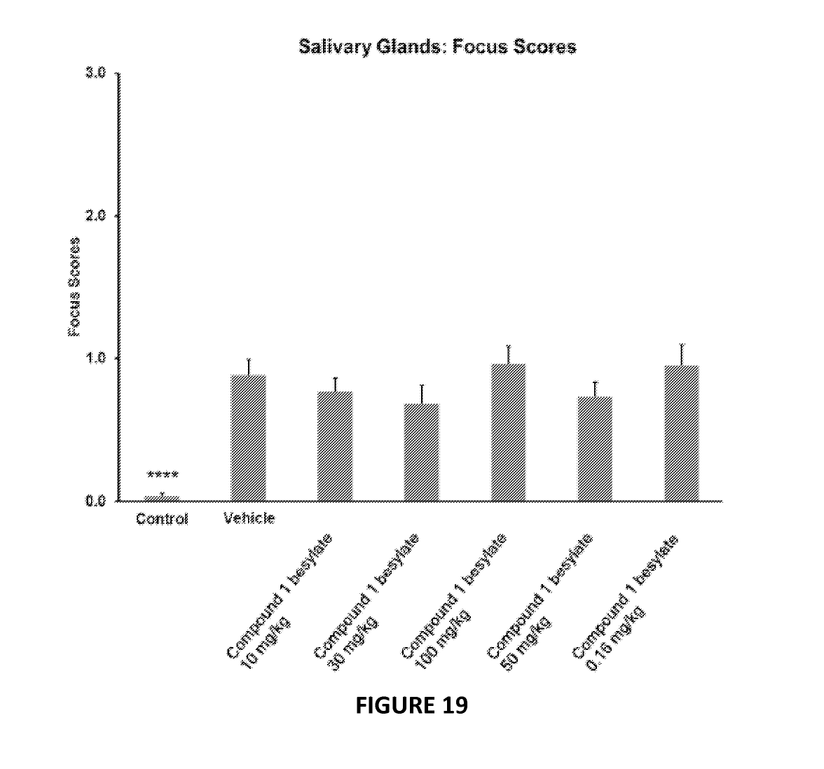

Focus Score data were analysed by Kruskal-Wallis test for non-parametric values followed by Dunn's post-test for multiple comparisons to the vehicle-treated group. A highly significant increase in the number of inflammatory foci was observed in the vehicle-treated group of NOD mice at 20 weeks of age when compared to age-matched control BALB/c mice (p<0.0001). Compound 1 besylate administration did not reduce the number of inflammatory foci when compared to the vehicle-treated group (FIG. 19).

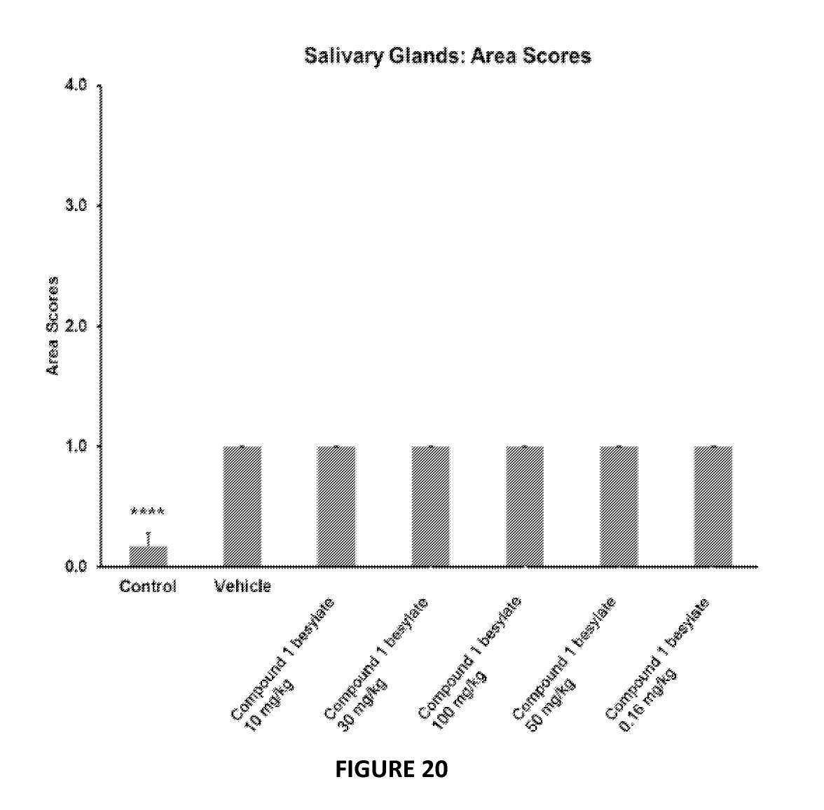

Area Score data were analysed by Kruskal-Wallis test for non-parametric values followed by Dunn's post-test for multiple comparisons to the vehicle-treated group. A highly significant increase in the proportion of infiltrated glandular tissue was observed in the vehicle-treated group of NOD mice at 20 weeks of age when compared to age-matched control BALB/c mice (p<0.0001). The percentage of tissue infiltrated in the vehicle-treated NOD mice was 45.8±6.4% when compared to 3.1±2.8% in the control group of BALB/c mice. Compound 1 besylate administration did not reduce the proportion of infiltrated glandular tissue when compared to the vehicle-treated group (FIG. 20).

Conclusion. Sjôgrens disease in NOD mice is characterised by clinical changes depending on the sex of the animals. In male mice the predominant tissue involved are the lachrymal glands, and there is also some salivary gland involvement which becomes more marked in older aged animals. In female mice the opposite profile is noted, with the salivary glands being the primary tissue targeted for immune attack. Studies typically use male mice as diabetes is less common in males and hence is less of a confounding factor in data interpretation and as a driver for group size. Clinical signs in NOD mice are variable and while they provide some indication of treatment efficacy, they only change markedly beyond 18 weeks. Histopathological changes are used to further validate the model and efficacy. This study was designed to be run until 20 weeks of age with histopathological changes as the primary readout for efficacy.

The data indicate that the NOD mice developed Sjogrens in the study. Some differences in lachrymal gland secretion rate were apparent when compared to control BALB/c mice and were starting to become marked by the chosen termination date. Most importantly, very clear evidence of lachrymal gland inflammation was apparent in the NOD control animals. Compound 1 besylate shows clear evidence of protecting against loss of lachrymal gland secretion. Animals treated with Compound 1 besylate at 30 mg/kg SID, 100 mg/kg SID or 50 mg/kg BID exhibited statistically significant increase in lachrymal gland secretions as compared to vehicle. Further, animals treated with Compound 1 besylate ad libitum at 0.16 mg/ml in the drinking water also exhibited statistically significant increase in lachrymal glad secretion. Salivary gland involvement, as assessed by measurement of secretion, showed no consistent change, as expected in this model at the timepoints tested. Such results demonstrate that treatment with Compound 1 besylate increases lachrymal gland secretion in NOD mice, which is indicative of a reduction in at least one of the symptoms of Sjogren's disease.

We claim:

1. A method of preventing, treating, stabilizing or lessening the severity or progression of an arthritic condition, the method comprising administering to a patient in need thereof a pharmaceutically acceptable composition comprising a therapeutically effective amount of N-(3-(5-fluoro-2-(4-(2-methoxyethoxyl)phenylamino)pyrimidin-4-ylamino)phenyl)acrylamide (1): 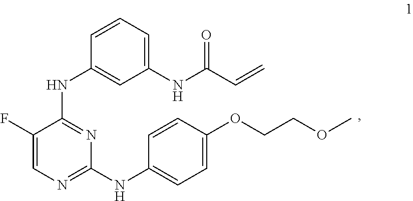 [SMILES: C=CC(=O)Nc1cccc(Nc2nc(Nc3ccc(OCCOC)cc3)ncc2F)c1]
or a pharmaceutically acceptable salt thereof.

2. The method according to claim 1, wherein the therapeutically effective amount of Compound 1 is about 125 mg BID to about 250 mg BID.

3. The method according to claim 1, wherein the therapeutically effective amount of Compound 1 is about 125 mg to about 375 mg.

4. The method according to claim 3, wherein the therapeutically effective amount of Compound 1 is about 125 mg QD.

5. The method according to claim 3, wherein the therapeutically effective amount of Compound 1 is about 375 mg.

6. The method according to claim 5, wherein Compound 1 is administered as two separate doses consisting of about 250 mg and about 125 mg.

7. The method according to claim 6, wherein the 375 mg dose of Compound 1 is administered according to the following dosing schedule:
(i) about 250 mg administered in the morning; and
(ii) about 125 mg administered in the evening.

8. The method according to claim 7, wherein the 250 mg dose is administered to a patient who has fasted prior to administration.

9. The method according to claim 7 or 8, wherein the 125 mg dose is administered H.S.

10. The method according to any of claims 1-9, wherein the arthritic condition is selected from osteoarthritis, rheumatoid arthritis, fibromyalgia, gout, ankylosing spondylitis, scleroderma, psoriatic arthritis, Sjogren's syndrome, Still's disease, Paget's disease, myositis, Lyme disease and juvenile idiopathic arthritis.

11. The method according to claim 10, wherein the arthritic condition is rheumatoid arthritis.

12. The method according to any of claims 1-11, wherein the pharmaceutically acceptable composition is formulated as an oral dosage form.

13. The method according to claim 1, wherein the pharmaceutically acceptable composition is administered twice a day.

14. The method according to any of claims 1-13, wherein the pharmaceutically acceptable composition is administered for at least one 28-day cycle.

15. The method according to any of claims 1-14, wherein Compound 1 is administered as a salt.

16. The method according to claim 15, wherein the salt is a benzenesulfonic acid salt.

17. The method according to claim 16, wherein the composition comprises from about 10% to about 50% N-(3-(5-fluoro-2-(4-(2-methoxyethoxyl)phenylamino)pyrimidin-4-ylamino)phenyl)acrylamide besylate.

18. The method according to claim 17, wherein the composition comprises about 42% N-(3-(5-fluoro-2-(4-(2-methoxyethoxyl)phenylamino)pyrimidin-4-ylamino)phenyl)acrylamide besylate.

19. The method according to claim 17 or claim 18, wherein the composition comprises from about 5% to about 15% by weight of wetting agent.

20. The method according to claim 19, wherein the composition comprises about 10% by weight of wetting agent.

21. The method according to claim 19 or 20, wherein the wetting agent is selected from poloxamer, polyoxyethylene ethers, polyoxyethylene sorbitan fatty acid esters polyoxyethylene fatty acid esters, polyethylene glycol fatty acid esters, polyoxyethylene hydrogenated castor oil, polyoxyethylene alkyl ether, polysorbates, cetyl alcohol, glycerol fatty acid esters, polyoxymethylene stearate, sodium lauryl sulfate, sorbitan fatty acid esters, sucrose fatty acid esters, benzalkonium chloride, polyethoxylated castor oil, and docusate sodium.

22. The method according to claim 21, wherein the wetting agent is a poloxamer.

23. The method according to claim 22, wherein the poloxamer is poloxamer 407.

24. The method according to any of claims 1-23, wherein the therapeutically effective amount is about 125 mg BID.

25. The method according to any of claims 1-23, wherein the therapeutically effective amount is about 250 mg BID.
